(19) United States
(12) Patent Application Publication
(10) Pub. No.: US 2008/0009506 A1
(43) Pub. Date: Jan. 10, 2008

(54) Imidazole Compound

(76) Inventors: Mari Kusama, Osaka-shi (JP); Tatsuya Watanabe, Osaka-shi (JP); Toshihiro Hosaka, Osaka-shi (JP); Yuko Kubota, Osaka-shi (JP)

Correspondence Address: BIRCH STEWART KOLASCH & BIRCH, PO BOX 747, FALLS CHURCH, VA, 22040-0747, US

(21) Appl. No.: 11/662,948

(22) PCT Filed: Sep. 16, 2005
(86) PCT No.: PCT/JP05/17540

§ 371 Date: Mar. 15, 2007

Related U.S. Application Data
(60) Provisional application No. 60/615,589, filed on Oct. 5, 2004.

(30) Foreign Application Priority Data
Sep. 17, 2004 (JP) 2004-270603
Oct. 4, 2004 (JP) 2004-291188
Dec. 1, 2004 (JP) 2004-348137
Jun. 30, 2005 (JP) 2005-191064

(51) Int. Cl. A61K 31/4178; A61K 31/415; A61K 31/4155; A61K 31/42; A61K 31/44; A61K 31/47; A61K 31/506; A61P 13/00; C07D 215/12; C07D 403/14; C07D 403/04; C07D 401/04; C07D 277/30; C07D 263/08; C07D 235/02; C07D 233/60; C07D 233/56; C07D 233/00; C07D 513/04; C07D 413/04; A61P 9/00; A61P 11/06; A61K 31/497; A61K 31/4439; A61K 31/425; A61K 31/4184; A61K 31/4168

(52) U.S. Cl. 514/255.05; 514/275; 514/301; 514/314; 514/341; 514/365; 514/374; 514/393; 514/397; 514/398; 514/399; 544/331; 544/405; 546/114; 546/165; 546/176; 546/274.1; 548/204; 548/237; 548/239; 548/302.7; 548/311.4; 548/311.7; 548/313.4; 548/314.7; 548/315.1; 548/338.1; 548/341.5; 548/343.5

(57) ABSTRACT

An imidazole compound of the formula: 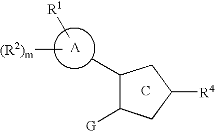 wherein Ring A is benzene or a heterocyclic ring; G is alkylthio, alkylsulfonyl, optionally substituted phenyl, or optionally substituted heterocyclic ring group, etc.; Ring C is imidazole;
R¹ is carbamoyl, etc.;
R² is cyano, nitro, hydroxyl, an alkoxy, a halogen, carboxy, an alkoxycarbonyl, carbamoyl, amino, an alkyl, etc.; m is 0 to 2; and
R⁴ is hydrogen, an alkyl, etc., or a pharmaceutically acceptable salt thereof, is a large conductance calcium-activated K channel opener useful for treatment of pollakiuria, urinary incontinence, etc.

### TECHNICAL FIELD

The present invention relates to a large conductance calcium-activated K channel opener, which is useful for treatment of diseases such as pollakiuria, urinary incontinence, asthma, chronic obstructive pulmonary diseases (COPD), cerebral infarction, subarachnoid hemorrhage and the like.

### BACKGROUND ART

Potassium is the most abundant intracelluar cation, and is very important in maintaining physiological homeostasis. Potassium channels are present in almost all vertebrate cells, and the potassium influx through these channels is indispensable for maintaining hyperpolarized resting membrane potential.

Large conductance calcium activated potassium channels (also BK channels or maxi-K channels) are expressed especially in neurons and smooth muscle cells. Because both of the increase of intracellular calcium concentration and membrane depolarization can activate maxi-K channels, maxi-K channels have been thought to play a pivotal role in regulating voltage-dependent calcium influx. Increase in the intracellular calcium concentration mediates many processes such as release of neurotransmitters, contraction of smooth muscles, cell growth and death, and the like. Actually, the opening of maxi-K channels causes strong membrane hyperpolarization, and inhibits these calcium-induced responses thereby. Accordingly, by inhibiting various depolarization-mediated physiological responses, a substance having an activity of opening maxi-K channels is useful for the treatment of diseases such as cerebral infarction, subarachnoid hemorrhage, pollakiuria, urinary incontinence, and the like.

There has been a report that a medicine which opens a BK channel has an activity to inhibit electrically induced contraction of respiratory tract preparation of guinea pig (J. Pharmacol. Exp. Ther., (1998) 286: 952-958)). Therefore, it is effective for treatment of, for example, asthma, COPD, etc. Also, there has been suggested that a medicine which opens a BK channel can be an agent for treatment of sexual function disorder such as erectile dysfunction, etc. (WO 00/34244).

There have been various reports on a large conductance calcium-activated potassium channel opener. For example, a pyrrole derivative (WO 96/40634), a furan derivative (JP 2000-351773-A), a nitrogen-containing 5-membered ring derivative in which the nitrogen atom is substituted by phenyl or benzyl (WO 98/04135), a diphenyltriazole derivative (J. Med. Chem., Vol. 45, p. 2942-2952 (2002)), Celecoxib derivative, etc. (EP 1400243), a diphenylheterocyclic compound (JP 2000-516925-A), a nitrogen-containing 5-membered heterocyclic ring compound (WO 02/83111), etc.

Also, as an imidazole derivative, there have been known an imidazole compound useful as a herbicide (JP 8-501100-A), a 2,3,4-substituted imidazole compound useful as a PAF antagonist (JP 2-503679), a 1,2-substituted imidazolyl compound useful as a COX-2 inhibitor (JP 10-503211-A), an imidazole compound useful as a COX inhibitor (WO 2004/099130), a 4,5-substituted imidazole compound useful as an anti-inflammatory agent (WO 96/03387), a pyridylimidazole compound useful as a fungicide for agricultural and horticultural use (JP 9-124640-A), an imidazole-4-carboxamide derivatives useful as an agent for treatment of obesity (WO 03/040107), an imidazole-4-carboxylic acid alkyl ester (J. Org. Chem., 2004, 69, 8829-35), but there have been no report regarding a use of these compounds for a BK channel opener.

### DISCLOSURE OF THE INVENTION

An object of the present invention is to provide a compound having an excellent large conductance calcium-activated K channel opening activity, and useful for the treatment of diseases such as pollakiuria, urinary incontinence, asthma, COPD, cerebral infarction, subarachnoid hemorrhage, and the like, with less side effects.

The present inventors have studied intensively to solve the above-mentioned problem, and as a result, they have found that a compound of the formulae shown below has an excellent large conductance calcium-activated K channel opening activity, whereby they have accomplished the present invention.

That is, the present invention is described as follows.
1. An imidazole compound of the formula (1): 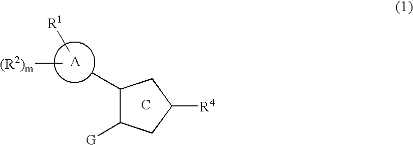
wherein Ring A is benzene or a heterocyclic ring;

G is —S(O)ₚ—R⁷, —O—R⁷, —N(R⁸)—R⁷ or

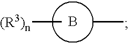

Ring B is benzene, a heterocyclic ring, a cycloalkane or a cycloalkene;

Ring C is a group selected from the following formulae: 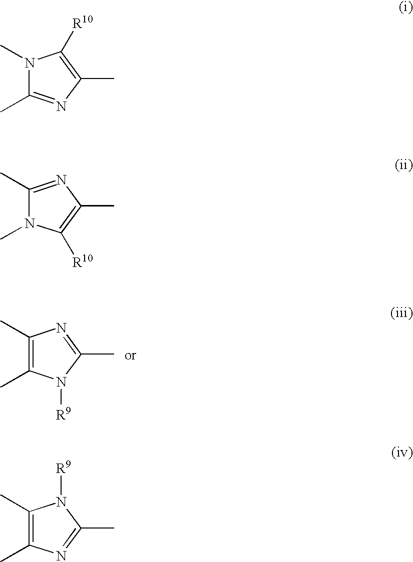
provided that when G is —S(O)ₚ—R⁷, —O—R⁷ or —N(R⁸)—R⁷, Ring C is a group of the formula (i);

R¹ is a group selected from the following formulae:

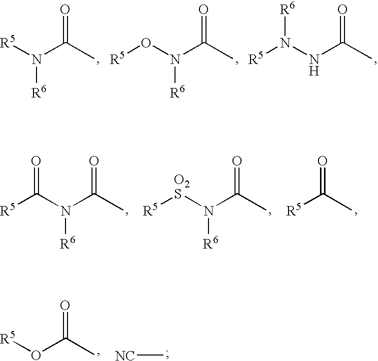

R² and R³ may be the same or different from each other, and each is cyano, nitro, hydroxyl, an optionally substituted alkoxy, a halogen, an alkanoyl, carboxy, an alkoxycarbonyl, a heterocyclic group, an optionally substituted carbamoyl, an optionally substituted amino or an optionally substituted alkyl; provided that when m is 2, two R²s may be the same or different from each other, and when n is 2, two R³s may be the same or different from each other;

m and n may be the same or different from each other, and each is 0, 1 or 2;

R⁴ is hydrogen, an alkoxy, an optionally substituted amino, an optionally substituted alkyl, an alkoxycarbonyl, an optionally substituted carbamoyl, carboxy, formyl or an optionally substituted heterocyclic group;

R⁵ and R⁶ may be the same or different from each other, and each is hydrogen, an optionally substituted alkyl, an optionally substituted cycloalkyl (wherein the cycloalkyl may be fused with an aryl), an optionally substituted aryl, an optionally substituted heterocyclic group, or an alkoxycarbonyl, or R⁵ and R⁶ may form an optionally substituted heterocyclic ring in combination with atom(s) to which they are bonded,

R⁷ is an optionally substituted alkyl, an optionally substituted aryl or an optionally substituted heterocyclic group;

p is 0, 1 or 2;

R⁸ is hydrogen or an alkyl;

R⁹ is a hydrogen or an alkyl, or R⁴ and R⁹ may be combined to form an alkylene; and

R¹⁰ is hydrogen or an alkyl;

or a pharmaceutically acceptable salt thereof.

2. The imidazole compound of the formula (1) except the following compounds (a) to (c), or a pharmaceutically acceptable salt thereof:

Compound (a) which is the compound of the formula (1) wherein Ring C is the group of the formula (i);

G is the following formulae:

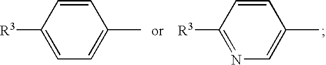

Ring A is the following formulae:

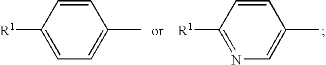

R¹ is halogen, cyano, alkoxycarbonyl, carbamoyl or carboxy;

R³ is alkoxy, hydroxy, amino, alkylamino or dialkylamino;

m is 0; n is 1; and
R⁴ is alkyl, halogen-substituted alkyl, hydroxyl-substituted alkyl, carbamoyl, N-alkylcarbamoyl, N,N-dialkylcarbamoyl, formyl, carboxy or alkoxycarbonyl;
Compound (b) which is the compound of the formula (I) wherein Ring C is the group of the formulae (iii) or (iv);

G is the following formula

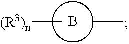

one of Ring A and Ring B is benzene, and the other is the following formula

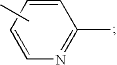

m and n may be the same or different from each other, and each is 0, 1 or 2;

R⁴ is hydrogen, amino or an alkyl optionally substituted by a halogen or an alkoxy;

R⁹ is hydrogen;

(i) when Ring A is benzene,

R¹ is a halogen, cyano or an alkoxycarbonyl;

R² is a halogen, an alkyl, an alkoxy, an haloalkyl, cyano, nitro, an haloalkoxy or an alkoxycarbonyl;

R³ is an alkyl, an alkoxy, an haloalkyl or a halogen;

(ii) when Ring B is benzene,

R¹ is a halogen;

R² is a halogen, an alkyl, an alkoxy or an haloalkyl;

R³ is a halogen, an alkyl, an alkoxy, an haloalkyl, cyano, nitro, an haloalkoxy or an alkoxycarbonyl; and
Compound (c): ethyl 2-(4-(ethoxycarbonyl)phenyl)-5-methyl-1-phenyl-1H-imidazole-4-carboxylate.
   3. An imidazole compound of the formula (1a) or a pharmaceutically acceptable salt thereof according to the above-mentioned 1 or 2: 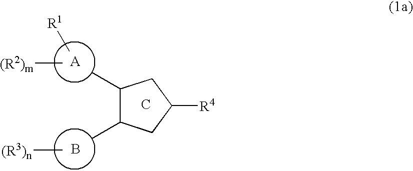
   wherein Ring A, Ring B, Ring C, R¹, R², R³, R⁴, m and n have the same meanings as defined above.
   4. The imidazole compound or a pharmaceutically acceptable salt thereof according to any one of the above-mentioned 1 to 3, wherein Ring A is benzene, pyridine, pyrimidine, thiazole, oxazole or thiophene.
   5. The imidazole compound or a pharmaceutically acceptable salt thereof according to any one of the above-mentioned 1 to 4, wherein Ring B is benzene, pyridine, pyrimidine, thiazole, thiophene, quinoline, pyrrole, benzo[b]thiophene, thieno[2,3-b]pyridine, thieno[3,2-b]pyridine, 1,4-benzodioxane, piperidine, oxazole or cyclohexene.
   6. The imidazole compound or a pharmaceutically acceptable salt thereof according to any one of the above-mentioned 1 to 4, wherein Ring B is a five-membered aromatic heterocyclic ring.
   7. The imidazole compound or a pharmaceutically acceptable salt thereof according to any one of the above-mentioned 1 to 4, wherein Ring B is thiophene.
   8. The imidazole compound or a pharmaceutically acceptable salt thereof according to the above-mentioned 1 to 3, wherein Ring A and Ring B may be the same or different from each other and each is benzene or pyridine.
   9. The imidazole compound or a pharmaceutically acceptable salt thereof according to any one of the above-mentioned 1 to 8, wherein R¹ is a group selected from the following formulae: 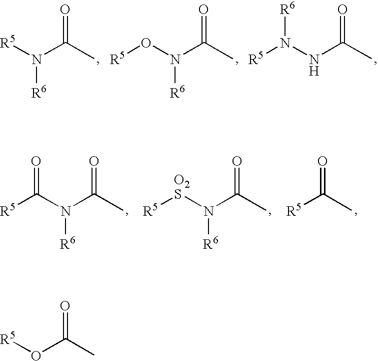
   wherein R⁵ and R⁶ have the same meanings as defined above.
   10. The imidazole compound or a pharmaceutically acceptable salt thereof according to any one of the above-mentioned 1 to 8, wherein R¹ is a group of the following formulae: 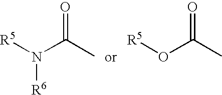
   wherein R⁵ and R⁶ have the same meanings as defined above.
   11. The imidazole compound or a pharmaceutically acceptable salt thereof according to any one of the above-mentioned 1 to 8, wherein R¹ is a group of the following formula:

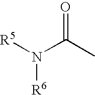

wherein R⁵ and R⁶ have the same meanings as defined above.
12. The imidazole compound or a pharmaceutically acceptable salt thereof according to any one of the above-mentioned 1 to 11, wherein R⁶ is hydrogen, an alkoxycarbonyl or an alkyl which may be substituted by hydroxy or alkoxy, and R⁵ is hydrogen or an alkyl which may be substituted by the same or different 1 to 3 groups selected from the following formulae: 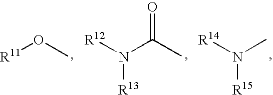
wherein R¹¹ is hydrogen, an alkyl or a hydroxyalkyl; R¹² and R¹³ may be the same or different from each other, and each is hydrogen, an alkyl, a hydroxyalkyl or an alkoxyalkyl; and R¹⁴ and R¹⁵ may be the same or different from each other, and each is hydrogen, an alkyl, an alkoxycarbonyl, an alkanoyl or an optionally substituted heterocyclic group.
   13. The imidazole compound or a pharmaceutically acceptable salt thereof according to any one of the above-mentioned 1 to 12, wherein m and n may be the same or different from each other, and each is 0 or 1.
   14. The imidazole compound or a pharmaceutically acceptable salt thereof according to any one of the above-mentioned 1 to 13, wherein R² and R³ may be the same or different from each other, and each is an alkoxy, a halogen, an optionally substituted alkyl or an optionally substituted amino.
   15. The imidazole compound or a pharmaceutically acceptable salt thereof according to any one of the above-mentioned 1 to 14, wherein R⁴ is a substituted alkyl optionally substituted by 1 to 3 halogens.
   16. An imidazole compound of the formula (1a-1): 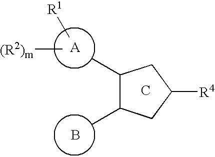
   wherein each symbol has the same meaning as defined above,
   or a pharmaceutically acceptable salt thereof.
   17. An imidazole compound of the formula (1a-2): 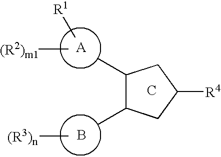
   wherein m1 is 1 or 2, and other symbols have the same meanings as defined above, or a pharmaceutically acceptable salt thereof.
   18. An imidazole compound of the formula (1a-3): 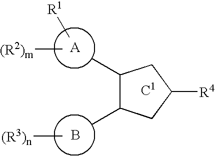
   wherein Ring C¹ is a group selected from the following formulae 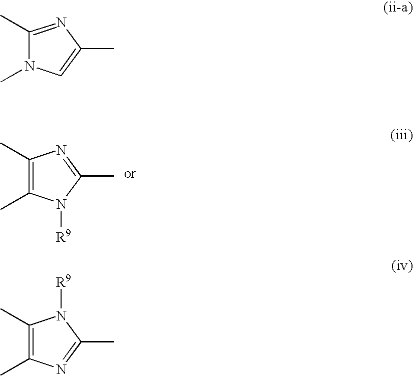
   wherein R⁹ has the same meaning as defined above, and
   other symbols have the same meanings as defined above,
   or a pharmaceutically acceptable salt thereof.
   19. An imidazole compound of the formula (1a-4) 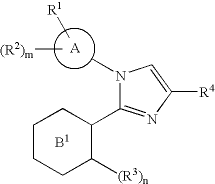
   wherein Ring B¹ is benzene or a 6-membered aromatic heterocyclic ring, and other symbols have the same meanings as defined above (Ring B¹ is preferably benzene or pyridine),
   or a pharmaceutically acceptable salt thereof.
   20. An imidazole compound of the formula (1a-5): 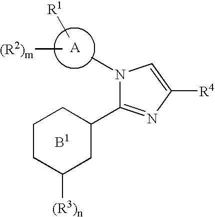
   wherein each symbol has the same meaning as defined above,
   (Ring B¹ is preferably benzene or pyridine),
   or a pharmaceutically acceptable salt thereof.
   21. An imidazole compound of the formula (1a-6) 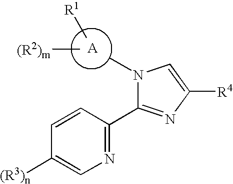
   wherein each symbol has the same meaning as defined above,
   or a pharmaceutically acceptable salt thereof.
   22. An imidazole compound of the formula (1a-7) 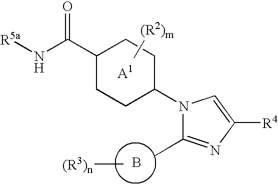
   wherein Ring A¹ is benzene or a 6-membered aromatic heterocyclic ring; R^{5a} is an optionally substituted alkyl, an optionally substituted cycloalkyl (the cycloalkyl may be fused with an aryl), an optionally substituted aryl, an optionally substituted heterocyclic group, or an alkoxycarbonyl; and other symbols have the same meanings as defined above (Ring A¹ is preferably benzene or pyridine),
   or a pharmaceutically acceptable salt thereof.
   23. An imidazole compound of the formula (1-A) 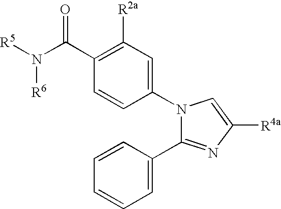
   wherein R^{2a} is a halogen, cyano, hydroxyl, an alkoxy, an amino optionally substituted by one or two alkyl(s), or an alkyl optionally substituted by 1 to 3 halogens; R^{4a} is an alkyl optionally substituted by 1 to 3 halogens; and other symbols have the same meanings as defined above,
   or a pharmaceutically acceptable salt thereof.
   24. An imidazole compound of the formula (1-B) 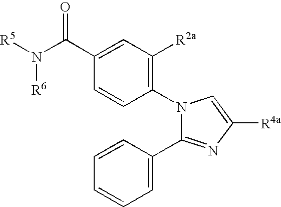
   wherein each symbol has the same meaning as defined above,
   or a pharmaceutically acceptable salt thereof.
   25. An imidazole compound of the formula (1-C) 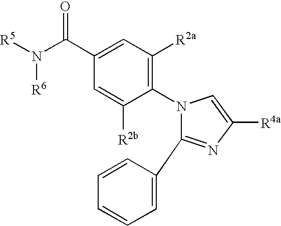
   wherein R^{2b} is a halogen, cyano, hydroxyl, an alkoxy, an amino optionally substituted by one or two alkyl(s), or an alkyl optionally substituted by 1 to 3 halogens; and other symbols have the same meanings as defined above,
   or a pharmaceutically acceptable salt thereof.
   26. An imidazole compound of the formula (1-D) 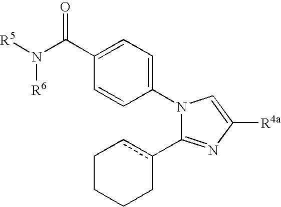
   wherein [symbol] represents single bond or double bond; and other symbols have the same meanings as defined above,
   or a pharmaceutically acceptable salt thereof.
   27. An imidazole compound of the formula (1-E) 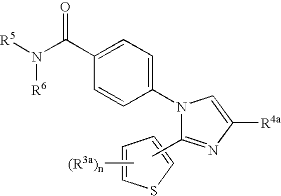
   wherein R^{3a} is a halogen, cyano, an alkanoyl, carboxy, an alkoxycarbonyl, or an alkyl optionally substituted by group(s) selected from an alkoxy, hydroxyl, a halogen and an amino optionally substituted by one or two alkyl(s); and other symbols have the same meanings as defined above, provided that when n is 2, two R^{3a}s may be the same or different,
   or a pharmaceutically acceptable salt thereof.
   28. An imidazole compound of the formula (1-F) 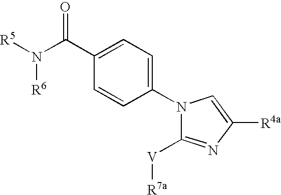
   wherein V is O, S(O)p or N(R⁸); R^{7a} is (1) an alkyl, (2) a phenylalkyl which may be substituted by 1 to 3 groups selected from the group consisting of an alkyl, an haloalkyl, a halogen and an alkoxy, or (3) a heterocyclic group-substituted alkyl which may be substituted by 1 to 3 groups selected from the group consisting of an alkyl, an haloalkyl, a halogen and an alkoxy, wherein the heterocyclic group is selected from pyridyl, pyrimidinyl and thienyl; and other symbols have the same meanings as defined above, or a pharmaceutically acceptable salt thereof.
   29. A medicine comprising the imidazole compound or a pharmaceutically acceptable salt thereof according to any one of the above-mentioned 1 to 28.
   30. The medicine according to the above-mentioned 29, which is a large conductance calcium-activated K channel opener.
   31. The medicine according to the above-mentioned 29, which is for the prophylaxis and/or treatment of pollakiuria, urinary incontinence, asthma or chronic obstructive pulmonary diseases.
   32. Use of the imidazole compound of the formula (1) except the compound wherein Ring C is the group of the formula (i); G is the following formulae: 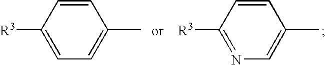
   Ring A is the following formulae: 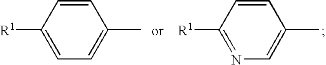
   R¹ is cyano, alkoxycarbonyl, carbamoyl or carboxy;
   R³ is alkoxy, hydroxy, amino, alkylamino or dialkylamino;
   m is 0; n is 1; and
   R⁴ is alkyl, halogen-substituted alkyl, hydroxyl-substituted alkyl, carbamoyl, N-alkylcarbamoyl, N,N-dialkylcarbamoyl, carboxy or alkoxycarbonyl;
   or a pharmaceutically acceptable salt thereof, in the preparation of a medicament for use in the treatment or prophylaxis of a disease against which a large conductance calcium-activated K channel opening activity is efficacious.
   33. Use of the imidazole compound or a pharmaceutically acceptable salt thereof according to any one of the above-mentioned 1 to 28, in the preparation of a medicament for use in the treatment or prophylaxis of a disease against which a large conductance calcium-activated K channel opening activity is efficacious.
   34. The use according to the above-mentioned 32 or 33, which is for the prophylaxis and/or treatment of pollakiuria, urinary incontinence, asthma or chronic obstructive pulmonary diseases.

36. The use according to any one of the above-mentioned 32 to 34, which is for the prophylaxis and/or treatment of pollakiuria, urinary incontinence or chronic obstructive pulmonary diseases.

### BEST MODE FOR CARRYING OUT THE INVENTION

Hereinafter, each group of the respective symbols in the present specification will be explained.

“Alkyl” is exemplified by a straight or branched C₁₋₆, preferably C₁₋₄ alkyl, more specifically by methyl, ethyl, propyl, isopropyl, butyl, isobutyl, 1-methylpropyl, pentyl, hexyl, etc.

“Hydroxyalkyl” is exemplified by a straight or branched C₁₋₆, preferably C₁₋₄ alkyl which is substituted by hydroxyl(s), more specifically by hydroxymethyl, 2-hydroxyethyl, 3-hydroxypropyl, 2-hydroxypropyl, 3-hydroxybutyl, 4-hydroxybutyl, etc.

“Alkoxy” and the alkoxy in “alkoxycarbonyl” are exemplified by a straight or branched C₁₋₆, preferably C₁₋₄ alkoxy, more specifically by methoxy, ethoxy, propoxy, isopropoxy, butoxy, isobutoxy, tert-butoxy, pentyloxy, hexyloxy, etc.

“Alkoxyalkyl” is exemplified by a straight or branched C₁₋₆, preferably C₁₋₄ alkyl which is substituted by a straight or branched C₁₋₆, preferably C₁₋₄ alkoxy, more specifically by methoxymethyl, ethoxymethyl, 2-methoxyethyl, 3-methoxypropyl, 2-methoxypropyl, 4-methoxybutyl, etc.

“Alkanoyl” is exemplified by a straight or branched C₁₋₆, preferably C₁₋₄ alkanoyl, more specifically by formyl, acetyl, propionyl, butyryl, pentanoyl, hexanoyl, etc.

“Halogen” is exemplified by fluorine, chlorine, bromine, and iodine.

“Aryl” is exemplified by a monocyclic, bicyclic or tricyclic C₆₋₁₄, preferably C₆₋₁₀ aryl, more specifically by phenyl, naphthyl, phenanthlyl, anthlyl, etc., particularly preferably by phenyl and naphthyl.

“Cycloalkyl” is exemplified by a C₃₋₈, preferably C₃₋₆ cycloalkyl, more specifically by cyclopropyl, cyclobutyl, cyclopentyl, cyclohexyl, etc. “Cycloalkyl fused with an aryl” is exemplified by a C₃₋₈, preferably C₃₋₆ cycloalkyl, which is fused with an aryl (preferably phenyl), more specifically by indanyl, tetranyl, etc. The “cycloalkyl” and the “cycloalkyl fused with an aryl” may have substituent(s) which are exemplified by hydroxyl, halogen, C₁₋₄ alkyl, C₁₋₄ alkoxy, etc., preferably by hydroxyl. Specific example of the substituted cycloalkyl fused with an aryl includes 2-hydroxyindan-1-yl, etc.

“Cycloalkane” is exemplified by a C₃₋₈, preferably C₃₋₆ cycloalkane, more specifically by cyclopropane, cyclobutane, cyclopentane, cyclohexane, etc., preferably cyclopropane, and cyclohexane.

“Cycloalkene” is exemplified by a C₃₋₈, preferably C₃₋₆ cycloalkene, more specifically by cyclopropene, cyclobutene, cyclopentene, cyclohexene, etc., preferably cyclohexene.

“Heterocyclic group” is exemplified by a monocyclic or bicyclic 5 to 10-membered heterocyclic group, which may be partially or wholly saturated, containing 1 to 4 hetero atom(s) selected from nitrogen, oxygen and sulfur. The monocyclic or bicyclic heterocyclic group which may be partially or wholly saturated may be substituted by oxo.

The monocyclic heterocyclic group is preferably exemplified by a 5 to 7-membered heterocyclic group which may be partially or wholly saturated, containing 1 to 4 hetero atom(s) selected from nitrogen, oxygen and sulfur, and it is specifically exemplified by oxazolyl, pyrrolidinyl, pyrrolyl, pyrazolyl, pyridyl, pyrimidinyl, pyrazinyl, tetrazolyl, thiazolyl, piperidyl, piperazinyl, morpholinyl, tetrahydropyranyl, tetrahydrofuryl, imidazolidinyl, oxazolidinyl, etc.

The bicyclic heterocyclic group is exemplified by a bicyclic heterocyclic group in which two of the same or different monocyclic heterocyclic groups above are fused, or a bicyclic heterocyclic group in which the above monocyclic heterocyclic group and benzene ring are fused, and it is specifically exemplified by dihydroindolyl, tetrahydroquinolyl, etc.

“Heterocyclic ring” of Ring A and Ring B is exemplified by a monocyclic or bicyclic 5 to 10-membered heterocyclic ring, which may be partially or wholly saturated, containing 1 to 4 hetero atom(s) selected from nitrogen, oxygen and sulfur, and preferably exemplified by a 5 or 6-membered aromatic heterocyclic ring. Specific examples thereof include thiophene, furan, pyrrole, thiazole, pyridine, pyrimidine, pyrazine, piperidine, piperazine, tetrahydropyran, benzo[b]thiophene, thieno[2,3-b]pyridine, thieno[3,2-b]pyridine, benzo[b]furan, 2,3-dihydroindole, 2,3-dihydrobenzo[b]furan, 1,4-benzodioxane, quinoline, 1,5-benzodioxepine, benzoxazoline, pyrrolopyridine, imidazopyridine, etc. Preferable heterocyclic ring in Ring A is exemplified by pyridine, pyrimidine, thiazole, oxazole and thiophene, particularly preferably pyridine. Preferable heterocyclic ring in Ring B is exemplified by pyridine, pyrimidine, thiazole, thiophene, quinoline, pyrrole, benzo[b]thiophene, thieno[2,3-b]pyridine, thieno[3,2-b]pyridine, piperidine, and 1,4-benzodioxane, more preferably pyridine, thiophene, pyrrole, piperidine, oxazole and 1,4-benzodioxane, particularly preferably pyridine and thiophene.

Bromine is preferable as the halogen of R¹.

“Heterocyclic ring formed by R⁵ and R⁶ in combination with atom(s) to which they are bonded” is exemplified by a saturated 5 to 8-membered monocyclic heterocycle which may have one or two hetero atom(s) (e.g. nitrogen, oxygen and sulfur, etc.). Specific examples thereof include pyrrolidine, piperidine, piperazine, morpholine, thiomorpholine, homopiperidine, etc.

The heterocyclic ring may be substituted, and the substituents are exemplified by (1) an alkyl which may be substituted by group(s) selected from (i) a halogen, (ii) hydroxyl, (iii) a haloalkoxy, (iv) an alkoxy which may be substituted by halogen(s), alkyl(s), phenyl(s), etc., (v) carbamoyl which may be substituted by alkyl(s), etc., (vi) cyano, (vii) an alkoxycarbonyl, (viii) carboxy, (ix) an amino which may be substituted by alkyl(s), phenyl(s), etc., and (x) an imino which may be substituted by an alkoxy, hydroxyl, etc.; (2) cyano; (3) a halogen; (4) an amino which may be substituted by alkyl(s), alkanoyl(s), cycloalkyl(s), etc.; (5) an alkenyl; (6) an imino which may be substituted by an alkoxy, hydroxyl, etc.; (7) a carbamoyl which may be substituted by alkyl(s), aralkyl(s), etc.; (8) an alkoxycarbonyl; (9) a heterocyclic group; etc. Preferred examples of the substituent(s) for the substituted heterocyclic ring include an alkyl substituted by hydroxyl(s), and a 5- or 6-membered monocyclic heterocyclic group which may have 1 to 3 hetero atom(s) selected from nitrogen, oxygen and sulfur. Specifically hydroxymethyl and pyrimidyl are preferred.

The substituent(s) for the substituted alkyl of R⁵ and R⁶ is exemplified by the group selected from the following formulae, etc., and the alkyl may be substituted by 1 to 3 same or different groups. 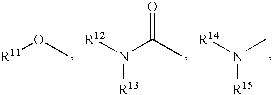
optionally substituted heterocyclic group
   wherein R¹¹, R¹², R¹³, R¹⁴ and R¹⁵ have the same meanings as defined above.

“Heterocyclic group” of R¹⁵, R⁶, R⁷ and R¹¹ to R¹⁵, and “heterocyclic group” which is the substituent for the substituted alkyl of R⁵, R⁶, R⁷ and R¹¹ to R¹⁵ are preferably exemplified by pyridyl, pyrazolyl, pyradinyl, pyrimidinyl, tetrazolyl, tetrahydropyranyl, thiazolyl, piperidine, etc. The substituent for the substituted heterocyclic group is exemplified by an alkyl, a haloalkyl, hydroxyl, an alkoxy, etc., preferably methyl, trifluoromethyl, hydroxyl, methoxy, etc. Particularly preferably example of the heterocyclic group of R¹⁴ and R¹⁵ is exemplified by pyridyl.

The substituent(s) for the substituted aryl of R⁵, R⁶ and R⁷, and the substituents for the substituted aryl which is the substituent for the substituted alkyl of R⁷ are exemplified by a halogen, hydroxyl, an alkoxy, an alkyl, a haloalkyl, etc.

The substituent(s) for the substituted carbamoyl of R², R³ and R⁴ are exemplified, respectively, by an alkyl which may be substituted by a halogen, hydroxyl, an alkoxy, amino, a mono- or dialkyl amino, etc.

The substituent(s) for the substituted amino of R², R³ and R⁴ are exemplified, respectively, by an alkyl which may be substituted by halogen(s), hydroxyl(s), alkoxy(s), alkoxycarbonyl(s), alkanoyl(s), amino(s) or mono- or dialkylamino(s), etc.

The substituent(s) for the substituted alkyl of R² and R³ are exemplified, respectively, by hydroxyl, an alkoxy, a halogen, an amino optionally substituted by one or two alkyl(s) etc., and specific examples of the substituted alkyl are exemplified by hydroxymethyl, 2-hydroxyethyl, methoxymethyl, trifluoromethyl, aminomethyl, etc.

The substituent(s) for the substituted alkoxy of R² and R³ are exemplified by a halogen, etc.

The substituent(s) for the substituted alkyl of R⁴ are exemplified by hydroxyl, an alkoxy, a halogen, etc., and specifically exemplified by hydroxymethyl, 2-hydroxyethyl, methoxymethyl, trifluoromethyl, etc.

Specific examples of the alkyl substituted by 1 to 3 halogen(s) of R⁴ are exemplified by trifluoromethyl, difluoromethyl, etc.

An oxazolyl is preferable as the heterocyclic group of R⁴.

The alkylene group formed by combination of R⁴ and R⁹ is exemplified by a C₃₋₆, preferably of C₃₋₅ alkylene, and specific examples are exemplified by trimethylene, tetramethylene, pentamethylene, etc.

The substituent(s) for the substituted alkyl of R⁷ are exemplified by an optionally substituted aryl or an optionally substituted heterocyclic group.

Examples of the pharmaceutically acceptable salts of compound (1) of the present invention may include, for example, inorganic acid salts such as hydrochloride, sulfate, phosphate or hydrobromide, and organic acid salts such as acetate, fumarate, oxalate, citrate, methanesulfonate, benzenesulfonate, tosylate or maleate, and the like. Also, in case of a compound having an acidic group such as carboxy, salts with a base (for example, alkali metal salts such as a sodium salt and a potassium salt, alkaline earth metal salts such as a calcium salt, organic base salts such as a triethylamine salt, or amino acid salts such as a lysine salt) can be mentioned.

Imidazole compound (1) or a pharmaceutically acceptable salt thereof includes any of its internal salts, and solvates such as hydrates.

In Compound (1) of the present invention, an optical isomer based on an asymmetric carbon may be present, and any of the isomers and a mixture thereof may be encompassed in the present invention. In addition, cis form and trans form may be present, in case that Compound (1) of the present invention has a double bond or a cycloalkanediyl moiety, and a tautomer may be present based on an unsaturated bond such as carbonyl, etc. in Compound (1) of the present invention, and any of these isomers and a mixture thereof may be encompassed in Compound (I) of the present invention.

Compound (I) of the present invention may be prepared by the following methods.

Further, unless otherwise specified, the following abbreviations in the present specification mean the following meanings, respectively.

DMF: dimethylformamide

THF: tetrahydrofuran

DMSO: dimethyl sulfoxide

DMA: dimethylacetamide

Bz: benzoyl

Me: methyl

Et: ethyl

ⁱPr: isopropyl

^{t}Bu: tertiary butyl
Method 1: The compound (1a) in which Ring C is group of the formula (i), and R⁴ is an optionally substituted alkyl or an alkoxycarbonyl may be prepared by the following method. 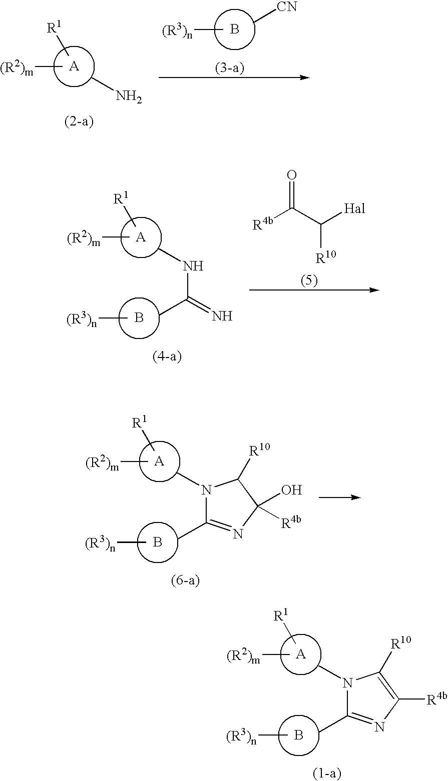
wherein Hal is a halogen (chlorine, bromine, etc.), R^{4b} is an optionally substituted alkyl or an alkoxycarbonyl, and other symbols have the same meanings as defined above.

The present reaction can be carried out in accordance with the method disclosed in J. Med. Chem., 1997, 40, 1634-1647, ibid., 2000, 43, 3168-3185 and Heterocycles 1995, 41(8), 1617-1620.

Compound (4-a) may be prepared by the following method.

(1) Compound (2-a) and Compound (3-a) are reacted in a suitable solvent (benzene, toluene, xylene, etc.) in the presence of an alkyl aluminum reagent (trimethylaluminum, triethylaluminum, dimethylaluminum chloride, diethylaluminum chloride, etc.) at 0 to 100° C. for 1 to 24 hours to give Compound (4-a).

(2) Compound (2-a) and Compound (3-a) are reacted in a suitable solvent (DMSO, DMF, 1,2-dimethoxyethane, THF, dioxane, etc.) in the presence of a base (sodium hydride, potassium hydride, sodium methoxide, sodium ethoxide, n-butyl lithium, lithium diisopropylamide, sodium hexamethyldisilazide, etc.) at −78° C. to a refluxing temperature of the solvent for 1 to 24 hours to give Compound (4-a).

Compound (4-a) is reacted with Compound (5) in a suitable solvent (methanol, ethanol, isopropyl alcohol, acetone, DMF, DMSO, etc.) in the presence of a base (sodium hydrogen carbonate, potassium hydrogen carbonate, sodium carbonate, potassium carbonate, triethylamine, diisopropylethylamine, etc.) at 0 to 100° C. for 1 to 24 hours to give Compound (6-a).

Compound (6-a) is treated with an acid catalyst (p-toluenesulfonic acid, etc.) in a suitable solvent (benzene, toluene, xylene, etc.) at a refluxing temperature of the solvent for 1 to 4 days to give Compound (1-a).

Compound (1-a) may directly be prepared by reacting Compound (4-a) with Compound (5) under the above-mentioned reaction condition.
Method 2: The compound (1a) in which Ring C is group of the formula (ii) and R⁴ is an optionally substituted alkyl or an alkoxycarbonyl may be prepared by the following method.

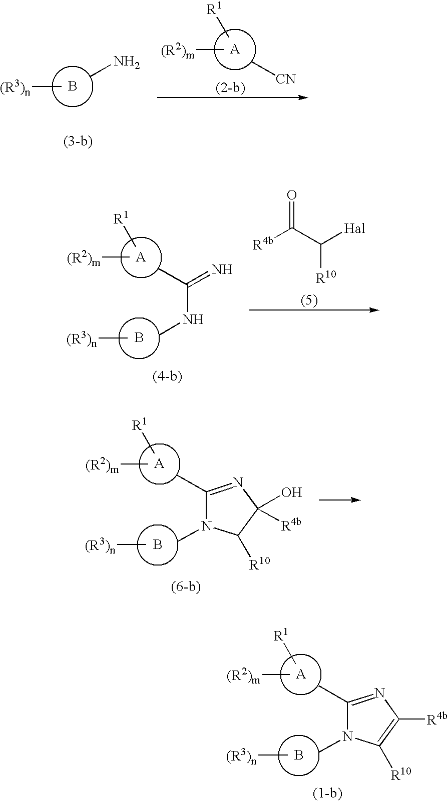

wherein the respective symbols have the same meanings as defined above.

This reaction can be carried out in the same manner as in Method 1.
Method 3: Compound (1a) may also be prepared by the following method. 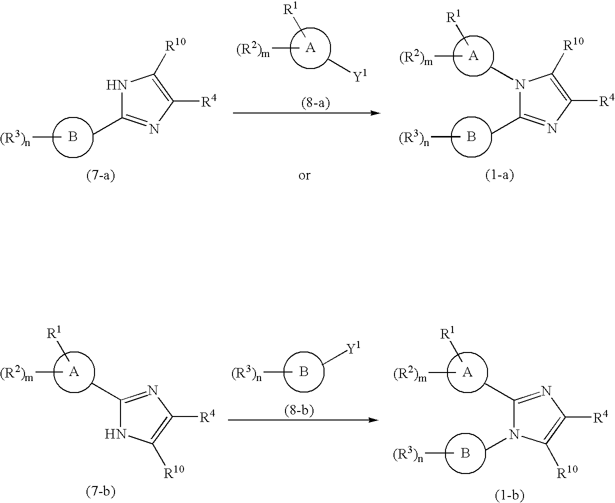
wherein Y¹ is B(OH)₂ or a halogen (fluorine, chlorine, bromine, etc.), and other symbols have the same meanings as defined above.

The reaction of Compound (7-a) and Compound (8-a), and the reaction of Compound (7-b) and Compound (8-b) may be carried out by the following method.

(1) When Y¹ is B(OH)₂, in accordance with the method described in Tetrahedron Letters 39 (1998), 2941-2944 and Organic Letters 2000, 2(9), 1233-1236, by reacting in a suitable solvent (chloroform, methylene chloride, THF, dioxane, DMF, etc.) in the presence of a copper catalyst (copper (II) acetate, [Cu(OH)TMEDA]₂Cl₂, etc.) preferably at room temperature for 1 to 24 hours, Compound (1-a) or Compound (1-b) can be prepared, respectively.

(2) When Y¹ is a halogen, by reacting in a suitable solvent (diethyl ether, THF, DMF, DMSO, methylene chloride, chloroform, etc.) in the presence of a base (sodium hydride, potassium hydride, lithium diisopropylamide, n-butyllithium, etc.) at ice-cooling temperature to 100° C. for 1 to 24 hours, Compound (1-a) or Compound (1-b) can be prepared, respectively.
Method 4: The Compound (1) in which R¹ is —CN, —COOR⁵ or —CONR⁵R⁶ may be prepared by the following method.

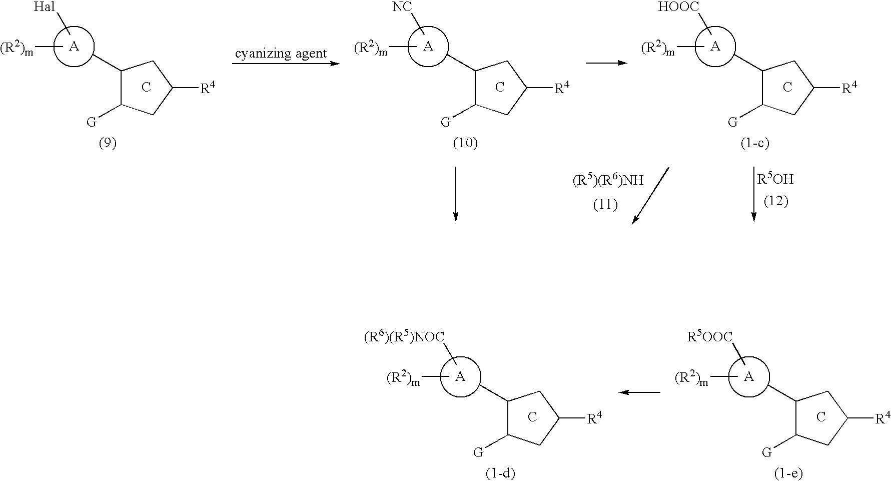

wherein the respective symbols have the same meanings as defined above.

Compound (9) is reacted with a cyanizing agent (sodium cyanide, cuprous cyanide, zinc cyanide, etc.) in a solvent (acetonitrile, DMSO, DMF or a mixture thereof) at room temperature to 100° C. for 1 to 24 hours to give Compound (10). Also, by using a tetrakis(triphenylphosphine) palladium catalyst, etc., and using a cyanizing agent such as zinc cyanide, potassium cyanide, etc., Compound (10) can be prepared.

Compound (10) is hydrolyzed by an acid (hydrochloric acid, sulfuric acid, etc.) or an alkali (sodium hydroxide, potassium hydroxide, etc.) in a solvent (water, methanol, ethanol, isopropyl alcohol, tert-butyl alcohol, ethylene glycol, diethylene glycol or a mixture thereof, etc.) to give Compound (1-c). The reaction temperature of this reaction is usually room temperature to 150° C., and the reaction time is usually 30 minutes to 48 hours.

Compound (1-d) or Compound (1-e) may be prepared, respectively, by either the following methods.

(1) Compound (1-c) is converted into an acid halide by treating with a halogenating agent (thionyl chloride, etc.) and the acid halide is reacted with Compound (11) or Compound (12) in the presence of a base (sodium hydrogen carbonate, potassium carbonate, triethylamine, pyridine, etc.) at −20° C. to room temperature for 30 minutes to 24 hours to give Compound (1-d) or Compound (1-e), respectively.

(2) Compound (1-c) is condensed with Compound (11) or Compound (12) in the presence of a condensing agent (1,3-dicyclohexylcarbodiimide, 1-ethyl-3-(3-dimethylaminopropyl) carbodiimide, carbonyldiimidazole, diethyl cyanophosphate, etc.) and, if necessary, in a solvent (DMF, THF, dioxane, etc.) to give Compound (1-d) or Compound (1-e), respectively. The reaction temperature is usually at 0° C. to 100° C. and the reaction time is usually for 30 minutes to 24 hours. In the reaction using the condensing agent, it may be carried out in the presence of 1-hydroxybenzotriazole, N-hydroxysuccinimide, etc., if necessary.

(3) Compound (1-c) is converted into a carbonic acid monoalkyl ester (methyl carbonate ester, ethyl carbonate ester, isobuthyl carbonate ester etc.), or a mixed acid anhydride with an organic acid (pivalic acid, isovaleric acid, etc.) and the resulting compound is condensed with Compound (11) or Compound (12) in a suitable solvent (THF, toluene, nitrobenzene or a mixed solvent thereof, etc.) in the presence of a base (triethylamine, pyridine, etc.) at −20° C. to room temperature for 1 to 24 hours to give Compound (1-d) or Compound (1-e), respectively.

Compound (9) or Compound (10) can be prepared by using a corresponding starting compound in accordance with Method 1 or Method 2.
Method 5: The compound (1) in which R¹ is —CON(R⁶)OR⁵ or —CONHN(R⁵)(R⁶) may be prepared by the following method.

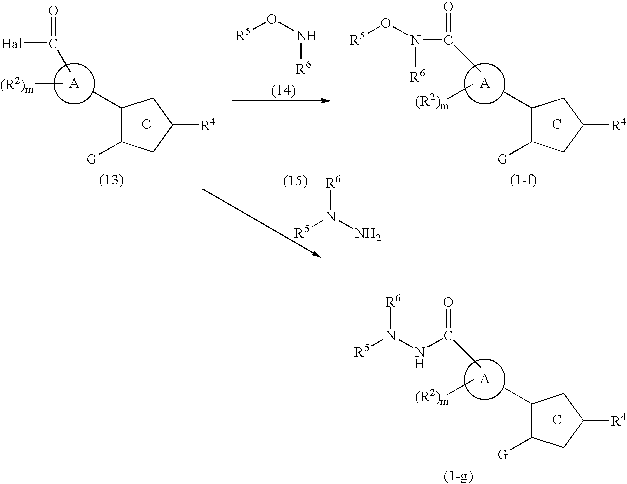

wherein the respective symbols have the same meanings as defined above.

Compound (13) is reacted with Compound (14) or Compound (15) in a suitable solvent (water, ethyl acetate, DMF, DMSO, chloroform, methylene chloride, THF or a mixture thereof, etc.) in the presence of a base (triethylamine, sodium hydrogen carbonate, potassium carbonate, etc.) at ice-cooling temperature to a refluxing temperature of the solvent for 1 to 24 hours to give Compound (1-f) or Compound (1-g).

Compound (13) can be prepared by using a corresponding starting compound in accordance with Method 4.
Method 6: The compound (1) in which R¹ is —COR⁵ may be prepared by the following method.

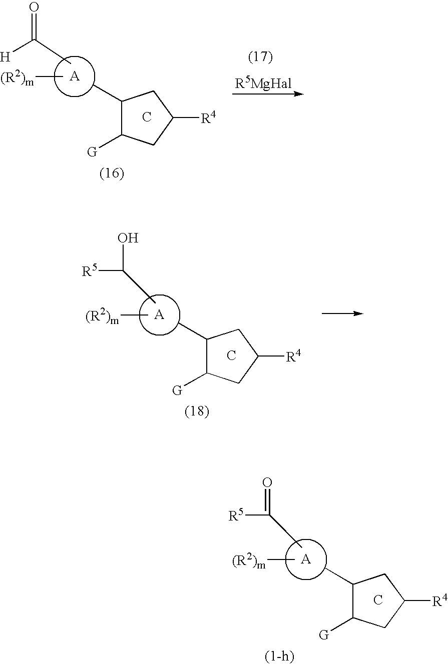

wherein the respective symbols have the same meanings as defined above.

Compound (16) is subjected to Grignard reaction with Compound (17) in a solvent (THF, diethyl ether, ethylene glycol dimethyl ether, benzene, toluene, xylene, dioxane, etc.) at −20 to 100° C. for 30 minutes to 24 hours to give Compound (18).

Compound (18) is reacted with an oxidizing agent [e.g. chromic acid-sulfuric acid, chromium (VI) oxide-sulfuric acid-acetone (Jones reagent), chromium (VI) oxide-pyridine complex (Collins reagent), dichromate (e.g. sodium dichromate, potassium dichromate, etc.)-sulfuric acid, pyridinium chlorochromate (PCC), manganese dioxide, DMSO-electrophilic activating reagent (e.g. dicyclohexylcarbodiimide, acetic anhydride, phosphorus pentaoxide, a sulfur trioxide-pyridine complex, trifluoroacetic anhydride, oxalyl chloride, and halogen), sodium hypochlorite, potassium hypochlorite, sodium bromite, etc.] at −20° C. to 100° C. for 30 minutes to 24 hours to give Compound (1-h).

Compound (16) can be prepared by using a corresponding starting compound in accordance with Methods 1 to 3.
Method 7: The compound (1) in which R¹ is —CON(R⁶)COR⁵ or —CON(R⁶)SO₂R⁵ may be prepared by the following method. 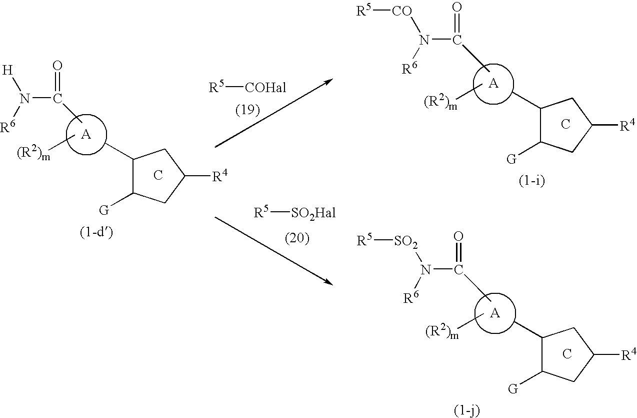
wherein the respective symbols have the same meanings as defined above.

Compound (1-d′) is reacted with Compound (19) or Compound (20) in the presence of a base (sodium hydrogen carbonate, potassium carbonate, triethylamine, pyridine, n-butyllithium, sodium hydride, sodium hydroxide, etc.) at −78° C. to 100° C. for 30 minutes to 24 hours to give Compound (1-i) or Compound (1-j).
Method 8: A compound in which R⁴ is an alkoxy and R¹⁰ is hydrogen in Compound (7-a) or Compound (7-b) may be prepared by the following method. 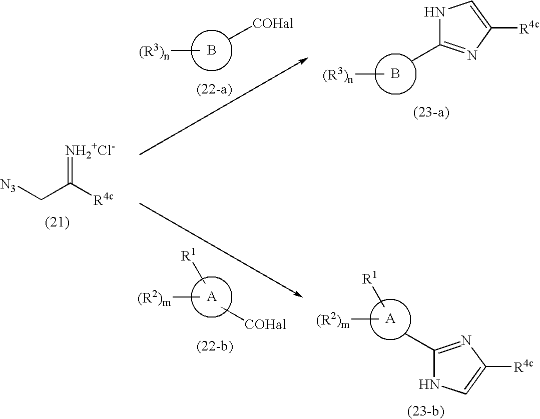
wherein R^{4c} is an alkoxy, and other symbols have the same meanings as defined above.

The present reaction can be carried out in accordance with the method described in Synthesis 1995, 449-452.

Compound (21) is treated with a base (triethylamine, diisopropylethylamine, pyridine, etc.) at room temperature, then reacting with triphenylphosphine in a suitable solvent (chloroform, methylene chloride, THF, dioxane, etc.) at room temperature, and further reacting with Compound (22-a) or Compound (22-b) to give Compound (23-a) or Compound (23-b), respectively.
Method 9: The compound (1a) in which R⁴ is an optionally substituted amino and R¹⁰ is hydrogen may be prepared by the following method. 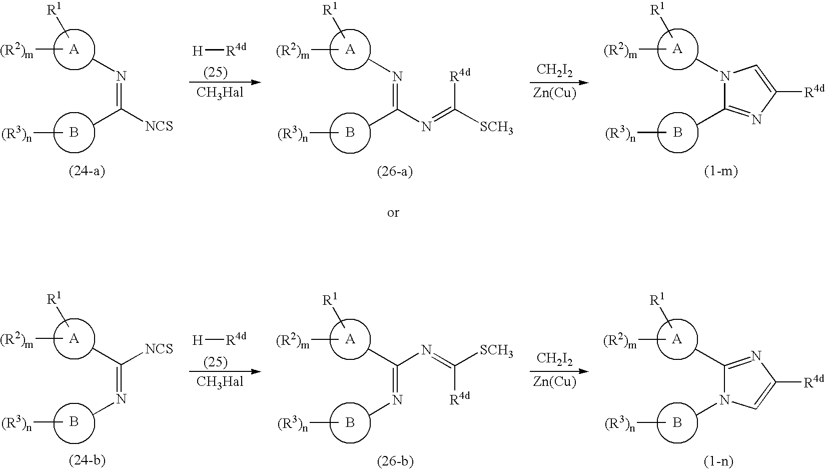
wherein R^{4d} is an optionally substituted amino, and other symbols have the same meanings as defined above.

In accordance with the method as described in Tetrahedron 51(27), 7459-7468, 1995, etc., Compound (24-a) or Compound (24-b) is reacted with Compound (25) in a suitable solvent (acetone, THF, dioxane, etc.) at room temperature to 100° C. for 1 to 12 hours, then reacting with a methyl halide for 1 to 12 hours to give Compound (26-a) or Compound (26-b), respectively.

Compound (24-a) or Compound (24-b) can be prepared in accordance with the method as described in Chem. Ber., 1968, 101, 3475-3490.

In accordance with the method as described in Tetrahedron 58 (2002), 2899-2904, Compound (26-a) or Compound (26-b) is reacted with a Simmons-Smith reagent in a suitable solvent (THF, dioxane, etc.) at a refluxing temperature of the solvent for 1 to 24 hours to give Compound (1-m) or Compound (1-n), respectively.
Method 10: The compound (1a) in which Ring C is the group of the formulae (iii) or (iv) may be prepared by the following method.

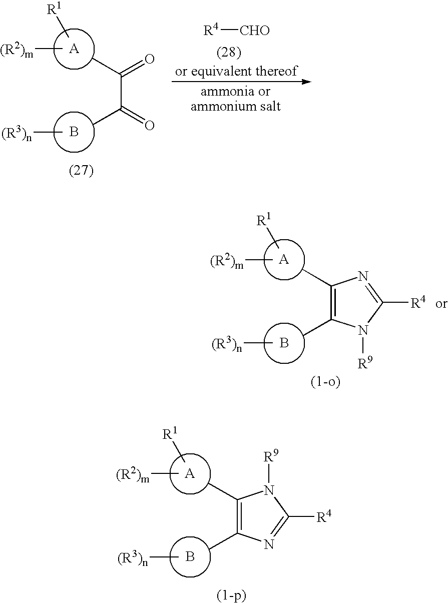

wherein the respective symbols have the same meanings as defined above.

(1) Preparation of the compound in which R⁹ is hydrogen

The present reaction can be carried out in accordance with the method described in Bioorg. Med. Chem. Lett., 1998, vol. 8, 3443-3448.

Compound (27) is reacted with Compound (28) or an equivalent thereof (acetal, hemiacetal, etc.) in a suitable solvent (acetic acid, methanol, ethanol, dimethoxyethane, THF, DMF, etc.) or without any solvent in the presence of ammonia or an ammonium salt (ammonium acetate, ammonium formate, ammonium carbonate, ammonium benzoate, ammonium picolate, etc.) at 0 to 150° C. for 1 to 48 hours to give the compound in which R⁹ is hydrogen.

Compound (27) can be prepared by using a corresponding starting compound in accordance with the method as described in Bioorg. Med. Chem. Lett., 1998, vol. 8, 3443-3448.

(2) Preparation of the compound in which R⁹ is an alkyl The compound in which R⁸ is an alkyl may be prepared by carrying out the method of (1) described above in the presence of alkylamine (methylamine, ethylamine, etc.).

Method 11: The compound (1a) in which Ring C is the group of the formulae (iii) or (iv), and R⁴ and R⁹ are combined to form an alkylene may be prepared by the following method. 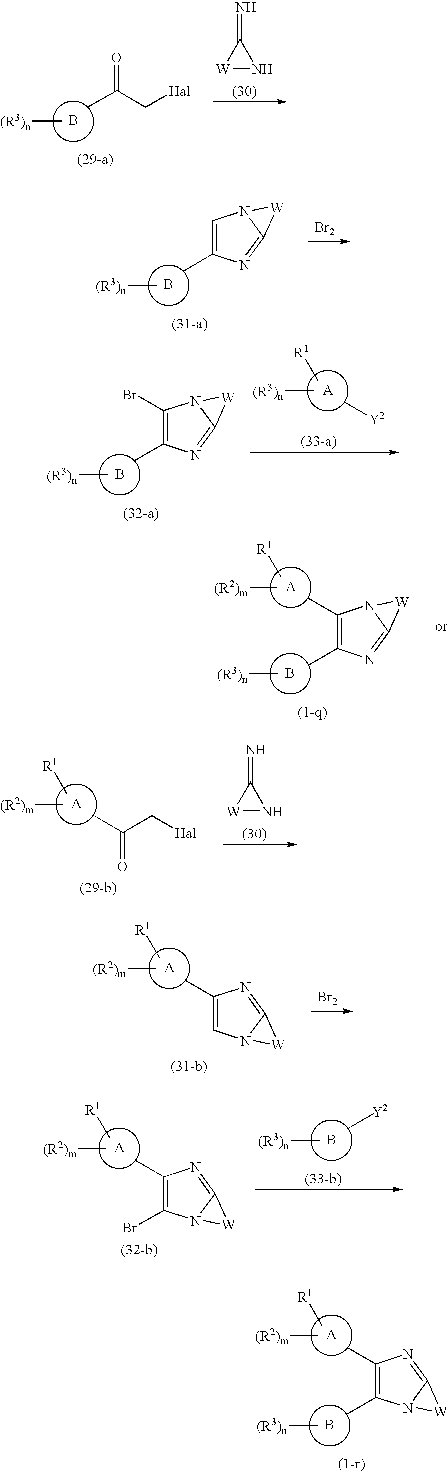
wherein Y² is —B(OH)₂, —B(OR^{a})₂ or —Sn(R^{a})₃, W is an alkylene, and other symbols have the same meanings as defined above.

The present reaction may be carried out in accordance with the method described in J. Med. Chem., 2002, 45, 999-1001 and JP 04-504709-A.

Compound (29-a) is reacted with Compound (30) in a suitable solvent (DMF, DMSO, DMA, etc.) in the presence of a base (sodium carbonate, potassium carbonate, sodium hydrogen carbonate, potassium hydrogen carbonate, triethylamine, etc.) at 0 to 100° C. for 1 to 24 hours to give Compound (31-a).

Compound (31-a) is treated with bromine in a suitable solvent (methylene chloride, chloroform, benzene, toluene, xylene, etc.) at ice-cooling temperature to room temperature for 1 to 24 hours to give Compound (32-a).

Compound (32-a) is reacted with Compound (33-a) in the presence of a palladium catalyst to give compound (1-q). A zerovalent or divalent palladium catalyst such as tetrakis (triphenylphosphine) palladium (0), bis(triphenylphosphine) palladium (II) chloride, palladium (II) acetate, etc., can be used as the palladium catalyst. In case of using the Compound (33-a) in which Y² is —B(OH)₂ or —B(OR)₂, it is preferable to add a base in the reaction. As a base, for example, an inorganic base such as alkali metal carbonate, alkali metal hydroxide, alkali metal phosphate and alkali metal fluoride, or organic base such as triethylamine can be used. Any solvent may be used as long as it has no adverse effect on the reaction, and examples of such solvent include dimethoxyethane, THF, dioxane, DMF, dimethylacetamide, toluene, benzene, water or a mixture thereof. The reaction temperature of the reaction is usually 60 to 150° C. and the reaction time is usually 1 to 24 hours.

Further, Compound (1-r) may be prepared by carrying out the above-mentioned methods using corresponding starting compounds.
Method 12: The compound (1) in which Ring C is the group of the formula i), G is —S(O)ₚ—R⁷ and R⁴ is an optionally substituted alkyl or an alkoxycarbonyl may be prepared by the following method. 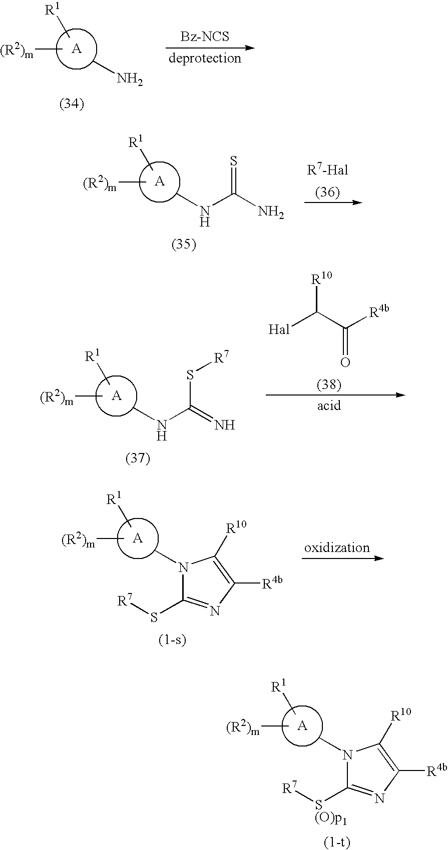
wherein p1 is 1 or 2, and other symbols have the same meanings as defined above.

The present reaction may be carried out in accordance with the method described in WO 01/64631. The reaction of Compound (34) and BzNCS may be carried out in a suitable solvent (THF, dioxane, diethyl ether, etc.) preferably at room temperature for 1 to 12 hours. The obtained reaction product is treated with a base (sodium hydroxide, potassium hydroxide, etc.) preferably at refluxing temperature for 30 minutes to 5 hours to give Compound (35).

The reaction of Compound (35) and Compound (36) may be carried out in a suitable solvent (acetone, methanol, ethanol, etc.) preferably at room temperature for 12 hours to two days.

The reaction of Compound (37) and Compound (38) may be carried out in a suitable solvent (methanol, ethanol, isopropyl alcohol, etc.) in the presence of a base (sodium hydrogen carbonate, sodium carbonate, potassium carbonate, triethylamine, etc.) preferably at refluxing temperature for 1 to 24 hours. The obtained reaction product may be treated with an acid (hydrochloric acid, sulfuric acid, p-toluenesulfonic acid, etc) in a suitable solvent (benzene, toluene, xylene, methanol, ethanol, etc.) preferably at refluxing temperature for 1 to 24 hours to give compound (1-s).

Compound (1-s) is reacted with an oxidizing agent (meta chloroperbenzoic acid, hydrogen peroxide, Oxone®), in a suitable solvent (acetic acid, dioxane, chloroform, methylene chloride, methanol, ethanol, isopropyl alcohol, butanol, water and a mixture thereof etc.) at 0 to 100° C. for 30 minutes to 24 hours to give compound (I-t).
Method 13: The compound (1a) in which Ring C is the group of the formulae (i) or (ii) may be prepared by the following method. 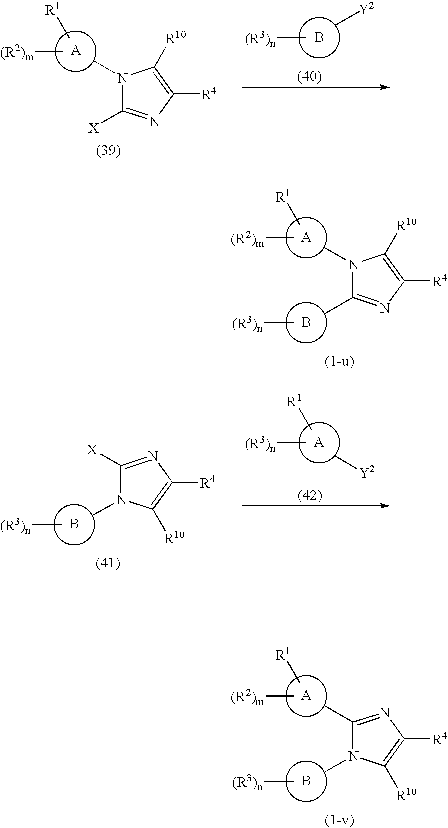
wherein X is a leaving group such as a halogen or an optionally substituted alkylsulfonyloxy (preferably trifluoromethanesulfonyloxy), Y² is —B(OH)₂, —B(OR^{a})₂ or —Sn(R^{a})₃, R^{a} is an alkyl and other symbols have the same meanings as defined above.

Compound (39) or Compound (41) may be reacted with Compound (40) or Compound (42), respectively, in the presence of a palladium catalyst to give Compound (1-u) or Compound (1-v). A zerovalent or divalent palladium catalyst such as tetrakis (triphenylphosphine) palladium (0), bis(triphenylphosphine) palladium (II) chloride, palladium (II) acetate, etc., can be used as the palladium catalyst. In case of using the Compound (40) or Compound (42) in which Y is —B(OH)₂ or —B(OR)₂, it is preferable to add a base in the reaction. As a base, for example, an inorganic base such as alkali metal carbonate, alkali metal hydroxide, alkali metal phosphate and alkali metal fluoride, or an organic base such as triethylamine can be used. Any solvent may be used as long as it has no adverse effect on the reaction, and examples of the solvent include DME, THF, dioxane, DMF, dimethylacetamide, toluene, benzene or a mixture thereof. The reaction temperature of the present reaction is usually 60 to 150° C., preferably 80 to 120° C. and the reaction time is usually 1 to 24 hours.

Compound (39) and Compound (41) can be prepared by converting the group S(O)p₁-R⁷ of Compound (1-t) and the corresponding compound into hydroxyl according to a conventional method, followed by halogenating or alkylsulfonylating. Also, Compound (1-t) and the corresponding compound may be used after the functional group of R⁴ is converted or modified according to a method described in the present specification or a conventional method, if necessary.
Method 14: The compound (1) in which Ring C is the group of the formula (I) and G is —S(O)p-R7, —O—R⁷ or —N(R⁸)—R⁷ may be prepared by the following method. 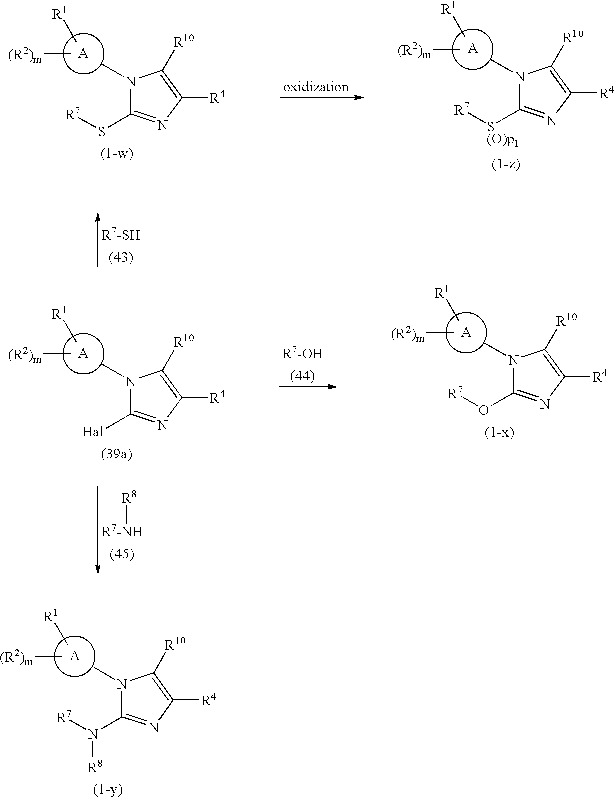
wherein the respective symbols have the same meanings as defined above.

Compound (39a) may be reacted with Compound (43), Compound (44) or Compound (45) in the presence of a base (sodium hydride, potassium hydride, etc.) in a solvent (THF, dioxane, DMF, DMSO, etc.) or without solvent to give Compound (1-w), Compound (1-x) or Compound (1-y), respectively. The reaction temperature is usually 0° C. to the refluxing temperature of the solvent, and the reaction time is usually 1 to 24 hours.

Compound (1-w) may be reacted with an oxidizing agent in accordance with Method 12 to give Compound (1-z).

In the above-mentioned methods, if the compound of the present invention, the intermediate compound, the starting compound, and the like have a functional group (hydroxyl, amino, carboxy, etc.), the reaction can proceed by protecting the functional group by a protecting group which is conventionally used in the field of synthetic organic chemistry, and after reaction, the protecting group is removed to give the desired compounds. The protecting groups for hydroxyl may be, for example, tetrahydropyranyl, trimethylsilyl, benzyl, and the like. The protecting groups for amino may be, for example, tert-butoxycarbonyl, benzyloxycarbonyl, and the like. The protecting groups for carboxy may be, for example, alkyl such as methyl and ethyl, and benzyl, and the like.

Further, after the compound of the present invention and the intermediate compound are prepared according to the above-mentioned methods, the functional group can be converted or modified according to the conventional method. More specifically, the following methods are mentioned.

(1) Modification of Amino

After an amino is protected if necessary, (i) a reaction with an alkyl halide, etc. may be carried out in the presence of a base (sodium hydride, triethylamine, sodium carbonate, potassium carbonate, etc.), or (ii) an alcohol, etc. may be subjected to Mitsunobu reaction with dialkyl azodicarboxylate and triphenylphosphine, and deprotection may be optionally carried out to convert the amino into a mono- or dialkylamino.

(2) Conversion of Amino into Amide

An amino may be converted into a corresponding amide by a reaction with an acyl halide.

(3) Conversion of Carboxy into Carbamoyl

Carboxy may be converted into a corresponding carbamoyl by a reaction with an amine.

(4) Hydrogenation of C═C Double Bond

A C═C double bond may be converted into a corresponding single bond by catalytic reduction using a transition metal (platinum, palladium, rhodium, ruthenium, nickel, etc.) catalyst.

(5) Hydrolysis of Ester

An ester may be converted into a corresponding carboxy by hydrolysis with an alkali (sodium hydroxide, potassium hydroxide, etc.).

(6) Conversion of Carbamoyl into Nitrile

Carbamoyl may be converted into a corresponding nitrile by a reaction with a dehydrating reagent (trifluoroacetic anhydride, etc).

(7) Conversion of Carboxy into 4,5-dihydrooxazol-2-yl

Carboxy may be converted into a corresponding 4,5-dihydrooxazol-2-yl by a reaction with 2-haloethylamine in the presence of a condensing agent.

(8) Halogenation or Alkylation of Hydroxyl

Hydroxyl may be converted into a corresponding halide by a reaction with a halogenating agent. Also, the halide may be converted into a corresponding alkoxy by a reaction with an alcohol.

(9) Reduction of Ester

Ester may be converted into a hydroxyl by a reduction with a reducing agent (a metal reducing reagent such as lithium aluminum hydride, sodium borohydride, lithium borohydride, etc.; diborane, etc.).

(10) Oxidation of Hydroxyl

Hydroxyl may be converted into an aldehyde, ketone or carboxy by oxidation.

(11) Amination of Ketone or Aldehyde

Ketone or aldehyde may be converted into a mono- or di-substituted aminomethyl by a reductive amination with an amine in the presence of a reducing agent (sodium borohydride, sodium cyanoborohydride, etc.).

(12) Conversion of Ketone or Aldehyde into Double Bond

Ketone or aldehyde may be converted into a double bond by Wittig reaction.

(13) Formation of Sulfoneamide Salt

Sulfoneamide may be converted into a corresponding sulfoneamide salt (a sodium salt, a potassium salt, etc.) by a treatment with sodium hydroxide, potassium hydroxide, etc. in an alcohol (methanol, ethanol, etc.).

(14) Conversion of Aldehyde into Oxime, Etc.

Aldehyde may be converted into a corresponding oxime, etc. by a reaction with hydroxylamine or O-alkyl hydroxylamine in the presence of a base (sodium hydrogen carbonate, etc.) in an alcohol (methanol, ethanol, etc.).

(15) Conversion of Halide into Nitrile

A halide may be converted into a corresponding nitrile by a reaction with a cyanizing agent.

(16) Amination Of Halide

A halide may be converted into a corresponding amine according to the method disclosed in Tetrahedron, 2002, p. 2041.

(17) Conversion of Carboxy into Carbamoyl or Hydroxymethyl

A carboxy may be converted into a corresponding carbamoyl by condensing with N-hydroxysuccinimide to give a succinimide ester, and then, reacting with an amine. Also, the succinimide ester may be converted into a corresponding hydroxymethyl by treating with a reducing agent (sodium borohydride, etc.).

(18) Dehalogenation

Dehalogenation of a halogen-substituted aromatic ring may be carried out by a reaction with potassium methoxide in the presence of a palladium catalyst in accordance with the methods described in Organometallics 2001, 20, 3607. Dehalogenation also may be carried out by catalytic reduction.

(19) Conversion of Carboxy into Amino

A carboxy may be converted into a corresponding amino by subjecting to the Curtius rearrangement reaction.

(20) Conversion into Difluoromethyl

Formyl may be converted into difluoromethyl by treating with DAST (Diethylaminosulfur trifluoride) in accordance with the method described in WO 01/64631.

(21) Halogenation of an Aromatic Ring

Halogenation of an aromatic ring may be carried out by reacting with halogenating agent (N-chlorosuccinimide, N-bromosuccinimide, etc).

(22) Conversion of Halogen into Alkoxy

A halogen may be converted into a corresponding alkoxy by reacting with alkali metal alkoxide (sodium methoxide, etc.).

(23) Conversion of Bromine into Nitrile

A bromine may be converted into a corresponding nitrile by reacting with potassium hexacyanoferrate (II) trihydrate in accordance with the methods described in J. Org. Chem., 2005, 70, 1508.

In the above-mentioned preparation methods, each of the prepared compounds and intermediates may be purified by the conventional method such as column chromatography, recrystallization, etc. Examples of the recrystallization solvent include an alcohol solvent such as methanol, ethanol, 2-propanol, etc., an ether solvent such as diethyl ether, etc., an ester solvent such as ethyl acetate, etc., an aromatic solvent such as toluene, etc., a ketone solvent such as acetone, etc., a hydrocarbon solvent such as hexane, etc., water, etc. or a mixed solvent thereof, etc. Also, the compound of the present invention may be converted into a pharmaceutically acceptable salt, and subsequently subjected to recrystallization, and the like.

The compound of the present invention or a pharmaceutically acceptable salt thereof may be prepared into a pharmaceutical composition comprising a therapeutically effective amount of the compound and a pharmaceutically acceptable carrier. The pharmaceutically acceptable carrier may include a diluent, a binder (e.g. syrup, Gum Arabic, gelatin, sorbit, tragacanth and polyvinyl pyrrolidone), an excipient (e.g. lactose, sucrose, corn starch, potassium phosphate, sorbit and glycine), a lubricant (e.g. magnesium stearate, talc, polyethylene glycol and silica), a disintegrator (e.g. potato starch) and a humectant (e.g. sodium lauryl sulfate).

The Compound of the present invention or a pharmaceutically acceptable salt thereof can be administered orally or parenterally, and used as suitable pharmaceutical preparations. As the suitable pharmaceutical preparation for oral administration, there are mentioned solid preparations such as tablets, granules, capsules and powders, or liquid preparations such as solutions, suspensions and emulsions. As the suitable pharmaceutical preparation for parenteral administration, there are mentioned a suppository, an injection or a drip infusion using distilled water for injection, physiological saline, an aqueous glucose solution, or an inhalant.

A dose of the compound of the present invention or a pharmaceutically acceptable salt thereof may vary depending on an administration route, an age, weight and condition of a patient, or a kind or degree of a disease, and may be generally about 0.1 to 50 mg/kg per day, particularly preferably about 0.1 to 30 mg/kg per day.

The compound of the present invention or a pharmaceutically acceptable salt thereof has an excellent large conductance calcium-activated K channel opening activity and hyperpolarizes a membrane electric potential of cells, and is useful for the prophylactic, relief and/or treatment for, for example, hypertension, premature birth, irritable bowel syndrome, chronic heart failure, angina, cardiac infarction, cerebral infarction, subarachnoid hemorrhage, cerebral vasospasm, cerebral hypoxia, peripheral blood vessel disorder, anxiety, male-pattern baldness, erectile dysfunction, diabetes, diabetic peripheral nerve disorder, other diabetic complication, sterility, urolithiasis and pain accompanied thereby, pollakiuria, urinary incontinence, nocturnal enuresis, asthma, chronic obstructive pulmonary diseases (COPD), cough accompanied by asthma or COPD, cerebral apoplexy, cerebral ischemia, traumatic encephalopathy, etc.

In the following, the present invention will be explained in detail by referring to Examples and Reference examples, but the present invention is not limited by these.

### EXAMPLE 1

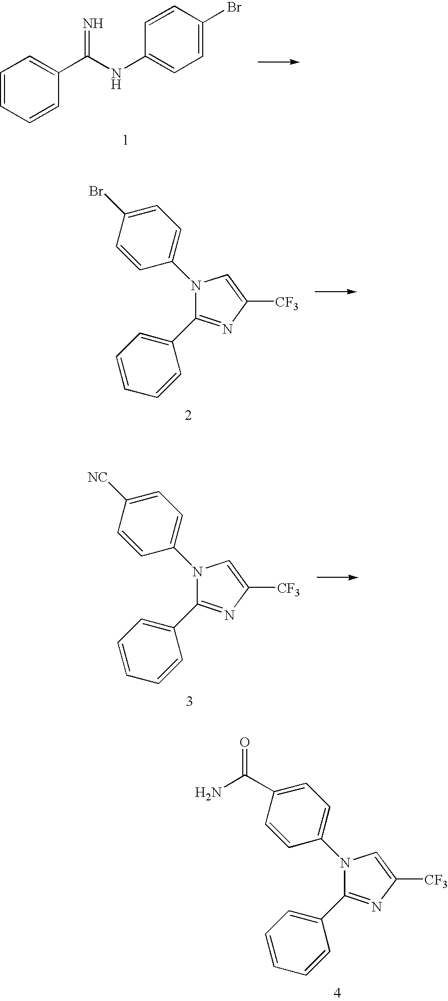 (1) An isopropyl alcohol (60 ml) suspension containing Compound 1 (1100 mg, 4.00 mmol), 3-bromo-1,1,1-trifluoroacetone (1.16 ml, 11.2 mmol) and sodium hydrogen carbonate (672 mg, 8.00 mmol) was refluxed under heating for 20 hours. The reaction mixture was poured into water and extracted with chloroform. The organic layer was separated, washed with brine, dried over anhydrous sodium sulfate and then concentrated under reduced pressure. To the obtained powder were added toluene (80 ml) and para-toluenesulfonic acid monohydrate (152 mg, 0.8 mmol), and the mixture was refluxed under heating for 26 hours. After cooling by allowing to stand, the reaction mixture was poured into water and extracted with chloroform. The organic layer was separated, washed with an aqueous sodium hydrogen carbonate solution and brine successively, dried over anhydrous sodium sulfate, and then concentrated under reduced pressure. The obtained residue was purified by silica gel column chromatography (hexane:ethyl acetate=9:1) to give Compound 2 (1179 mg, 80%) as powders.
MS: 367/369[M+H]⁺, APCI (MeOH)
(2) A DMF (7 ml) solution containing Compound 2 (551 mg, 1.50 mmol), zinc cyanide (176 mg, 1.50 mmol) and tetrakis(triphenylphosphine)palladium (173 mg, 0.15 mmol) was heated by a Microwave reaction device at 175° C. for 5 minutes. After cooling by allowing to stand, the reaction mixture was diluted by water and ethyl acetate, and filtered through Celite. The filtrate was extracted with ethyl acetate, the organic layer was washed with brine, dried over anhydrous sodium sulfate, and then concentrated under reduced pressure. The residue was purified by silica gel column chromatography (hexane:ethyl acetate=9:1) to give Compound 3 (379 mg, 81%) as powders.
MS: 314[M+H]⁺, APCI (MeOH)
(3) Compound 3 (80 mg, 0.254 mmol) and potassium hydroxide (powder) (143 mg, 2.54 mmol) were refluxed under heating in tert-butyl alcohol (2 ml) for 5 hours. After cooling by allowing to stand, the reaction mixture was poured into water and extracted with ethyl acetate. The organic layer was washed with brine, dried over anhydrous sodium sulfate, and then concentrated under reduced pressure. The residue was purified by silica gel column chromatography (chloroform:methanol=100:0->95:5) to give Compound 4 (40 mg, 48%) as powders.
MS: 332[M+H]⁺, APCI (MeOH)

### EXAMPLE 2

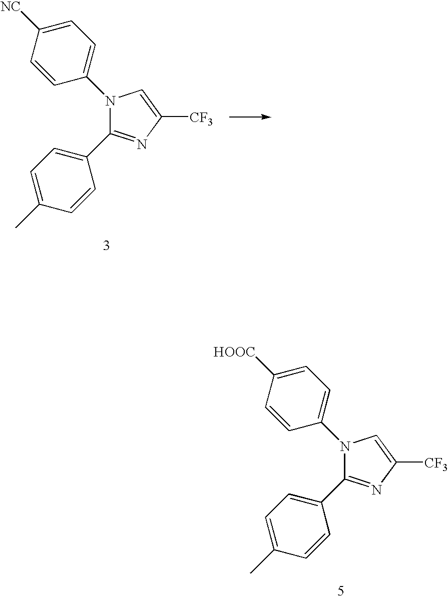

Compound 3 (774 mg, 2.36 mmol) and potassium hydroxide (powder) (685 mg, 12.2 mmol) were refluxed under heating in n-propanol (25 ml) for 24 hours. After cooling by allowing to stand and concentration, the reaction mixture was poured into water, and then a pH thereof was adjusted to 3 to 4 by adding 10% aqueous hydrochloric acid. The reaction mixture was extracted with ethyl acetate, the organic layer was washed with brine, dried over anhydrous sodium sulfate, and then, concentrated under reduced pressure. The residue was purified by silica gel column chromatography (chloroform:methanol=100:0->95:5) to give Compound 5 (702 mg, 86%) as powders.

MS: 345 [M−H]⁻, ESI (MeOH)

### EXAMPLE 3

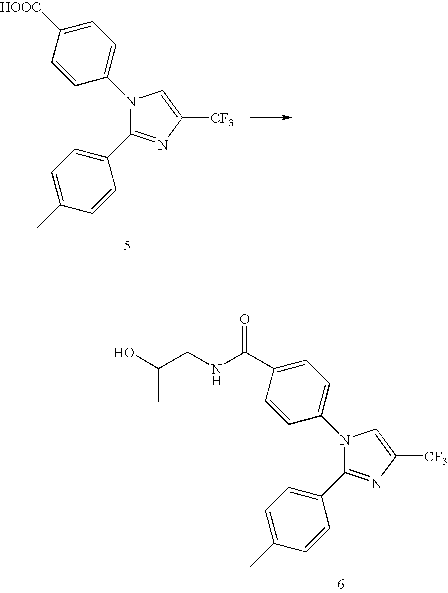

To a methylene chloride (20 ml) solution containing Compound 5 (640 mg, 1.85 mmol) were added oxalyl chloride (0.26 ml, 2.98 mmol) and DMF (2 drops) under ice-cooling. The mixture was stirred at room temperature for 6 hours and concentrated to give a crude carboxylic acid chloride. The obtained crude carboxylic acid chloride was used in the next reaction without purification. To a methylene chloride (3 ml) solution containing the crude carboxylic acid chloride (68 mg, 0.185 mmol) was added 1-amino-2-propanol (36 μl, 0.466 mmol) and the mixture was stirred for 24 hours. The reaction mixture was concentrated, and the obtained residue was purified by silica gel column chromatography (chloroform:methanol=100:0->95:5) to give Compound 6 (57 mg, 77%) as powders.

MS: 404[M+H]⁺, APCI (MeOH)

### EXAMPLE 4

The following compounds were prepared by subjecting to reaction and treatment in the same manner as in Example 1.

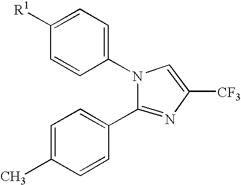

| Example | R¹ | MS |
|---|---|---|
| Example 4(1) | Br | 381/383[M+H]⁺, APCI |
| Example 4(2) | CN | 328[M+H]⁺, APCI |
| Example 4(3) | CONH₂ | 346[M+H]⁺, APCI |

### EXAMPLE 5

The following compounds were prepared by subjecting to reaction and treatment in the same manner as in Examples 1 and 2.

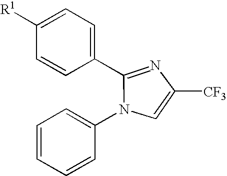

| Example | R¹ | MS |
|---|---|---|
| Example 5(1) | Br | 367/369[M+H]⁺, APCI |
| Example 5(2) | CN | 314[M+H]⁺, APCI |
| Example 5(3) | COOH | 331[M+H]⁻, ESI |

### EXAMPLES 6 TO 10

The following compounds were prepared by subjecting to reaction and treatment in the same manner as in Example 3.

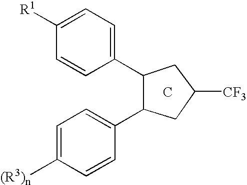

| Example | Ring C | R¹ | R³ | n | MS |
|---|---|---|---|---|---|
| Example 6 | 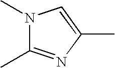 | CONH(CH₂)₂OCH₃ | CH₃ | 1 | 404[M+H]⁺, APCI |
| Example 7 | 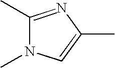 | 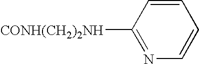 | +113 | 0 | 452[M+H]⁺, ESI |
| Example 8 | 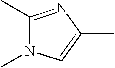 | CONH(CH₂)₂OH | — | 0 | 376[M+H]⁺, ESI |
| Example 9 | 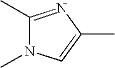 | 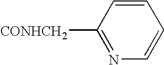 | — | 0 | 423[M+H]⁺, ESI |
| Example 10 | 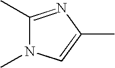 | 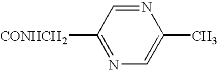 | — | 0 | 438[M+H]⁺, ESI |

Example 11

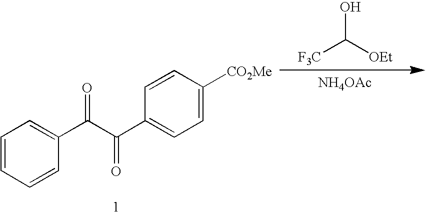

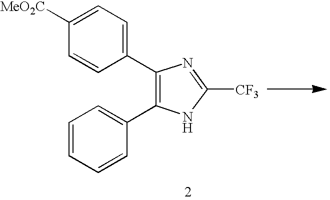

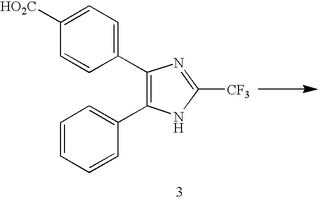

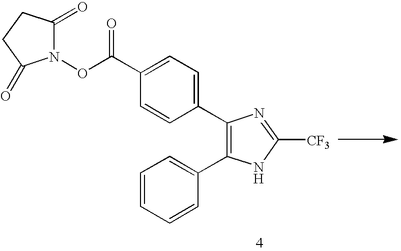

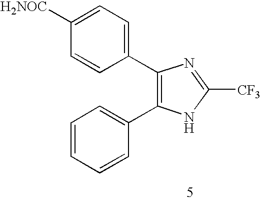

(1) An acetic acid (30 ml) suspension containing Compound 1 (1.62 g, 6.03 mmol), trifluoroacetaldehyde ethylhemiacetal (3.49 ml, 30.0 mmol) and ammonium acetate (2.31 g, 30.0 mmol) was refluxed under heating for 17 hours. After cooling the reaction mixture, it was poured into water (150 ml) and neutralized by 28% aqueous ammonia. The formed precipitates were collected by filtration, washed with water and air-dried at 80° C. The obtained crude crystals were recrystallized from ethanol, and further recrystallized from ethyl acetate to give Compound 2 (627 mg, 30%) as crystals.
MS: 347 [M+H]⁺, APCI
(2) To a methanol (10 ml) suspension containing Compound 2 (625 mg, 1.80 mmol) was added dropwise 2N aqueous sodium hydroxide solution (5 ml, 10.0 mmol), and the mixture was stirred at room temperature for 16 hours. The reaction mixture was concentrated under reduced pressure and the residue was washed with ether. The aqueous layer was neutralized with 1N hydrochloric acid (10 ml), and then, extracted with chloroform. The organic layer was dried over anhydrous sodium sulfate, and concentrated under reduced pressure to give a crude product (768 mg) of Compound 3.

To a DMF (15 ml) solution containing the obtained crude carboxylic acid (768 mg) and N-hydroxysuccinimide (373 mg, 3.24 mmol) was added under ice-cooling 1-ethyl-3-(3′-dimethylaminopropyl)carbodiimide hydrochloride (549 mg, 2.88 mmol), and the mixture was stirred at room temperature for 1.5 hours. The reaction mixture was diluted by ethyl acetate, washed with water and brine successively, dried over anhydrous sodium sulfate, and then, concentrated under reduced pressure. The residue was purified by silica gel column chromatography (ethyl acetate:chloroform=5:95->10:90) to give Compound 4 (682 mg, 88%) as powders.

MS: 430[M+H]⁺, APCI

(3) To a THF (3 ml) solution containing Compound 4 (100 mg, 0.233 mmol) was added under ice-cooling 28% aqueous ammonia (0.142 ml, 2.33 mmol), and the mixture was stirred at the same temperature for 1.5 hours. Water and hexane were added to the reaction mixture, and the mixture was extracted with ethyl acetate. The organic layer was washed with a saturated sodium bicarbonate solution and water, and dried over anhydrous sodium sulfate. After the solvent was removed under reduced pressure, the residue was triturated with ethyl acetate-diisopropyl ether-hexane to give Compound 5 (51 mg, 66%).

MS: 332 [M+H]⁺, APCI

### EXAMPLE 12

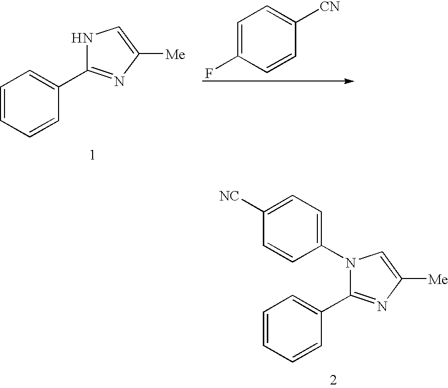

To a DMSO (20 ml) suspension containing sodium hydride (60% purity, 0.575 g, 14.4 mmol) was added Compound 1 (2.19 g, 13.8 mmol) little by little at room temperature, and the mixture was stirred at room temperature for an hour. To the mixture was added a DMSO (10 ml) solution containing 4-fluorobenzonitrile (1.67 g, 13.8 mmol), and the mixture was stirred at 120° C. for 24 hours. After cooling, the reaction mixture was poured into ice-water, and extracted with ethyl acetate. The organic layer was successively washed with water and brine, dried over anhydrous sodium sulfate, and then, concentrated under reduced pressure. The residue was purified by silica gel column chromatography (hexane:ethyl acetate=2:1->1:1) and triturated with diisopropyl ether to give Compound 2 (1.92 g, 54%) as powders.

MS: 260[M+H]⁺, APCI

### EXAMPLE 13

The following compounds were prepared by subjecting the compound of Example 12 to reaction and treatment in the same manner as in Example 1(3).

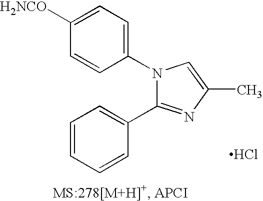

### EXAMPLES 14-39

The following compounds were prepared in accordance with the above mentioned Examples, methods disclosed in this specification and methods disclosed in the conventionally known documents.

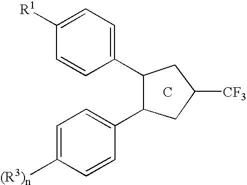

| Example | Ring C | R¹ | R³ | n | MS |
|---|---|---|---|---|---|
| Example 14 | 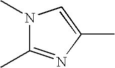 | CONH(CH₂)₂OH | — | 0 | 376[M+H]⁺, APCI |
| Example 15 | 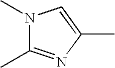 | CONH(CH₂)₂NHCOOMe | — | 0 | 433[M+H]⁺, APCI |
| Example 16 | 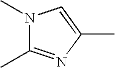 | CONH(CH₂)₂NHCOOMe | 4-Me | 1 | 477[M+H]⁺, APCI |
| Example 17 | 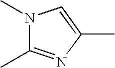 | CONH(CH₂)₂OH | 4-Me | 1 | 390[M+H]⁺, APCI |
| Example 18 | 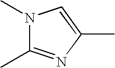 | CONHCH₂CONH₂ | 4-Me | 1 | 403[M+H]⁺, APCI |
| Example 19 | 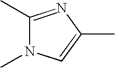 | CONH(CH₂)₂CH₃ | — | 0 | 374[M+H]⁺, APCI |
| Example 20 | 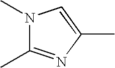 | 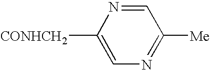 | — | 0 | 438[M+H]⁺, APCI |
| Example 21 | 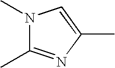 | CONHCH₂CH(OH)CH₃ (R) | — | 0 | 390[M+H]⁺, APCI |
| Example 22 | 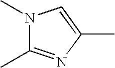 | CONHCH₂CH(OH)CH₂OH (S) | — | 0 | 406[M+H]⁺, APCI |
| Example 23 | 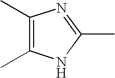 | CONHCH₂CH(OH)CH₃ (R) | — | 0 | 390[M+H]⁺, APCI |
| Example 24 | 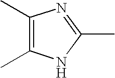 | 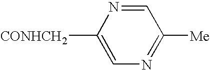 | — | 0 | 438[M+H]⁺, APCI |
| Example 25 | 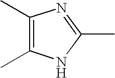 | CONH(CH₂)₂OH | — | 0 | 376[M+H]⁺, APCI |
| Example 26 | 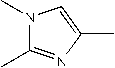 | CONH₂ | 2-Me | 1 | 346[M+H]⁺, APCI |
| Example 27 | 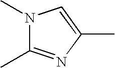 | CONH₂ | 3-Me | 1 | 346[M+H]⁺, APCI |

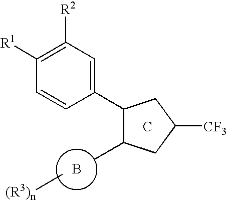

| Example | (R³)ₙ-Ring B | Ring C | R¹ | R² | MS |
|---|---|---|---|---|---|
| Example 28 | 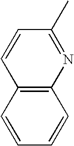 | 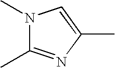 | CONH₂ | H | 383[M+H]⁺, APCI |
| Example 29 | 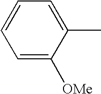 | 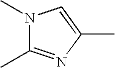 | CONH₂ | H | 362[M+H]⁺, APCI |
| Example 30 | 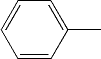 | 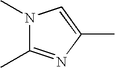 | 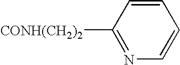 | H | 437[M+H]⁺, APCI |
| Example 31 | 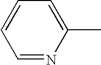 | 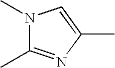 | CONH₂ | H | 333[M+H]⁺, APCI |
| Example 32 | 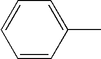 | 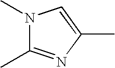 | CONH₂ | Me | 346[M+H]⁺, APCI |
| Example 33 | 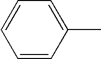 | 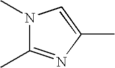 | CN | Me | 328[M+H]⁺, APCI |
| Example 34 | 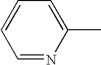 | 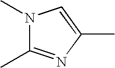 | Br | H | 368/370[M+H]⁺, APCI |
| Example 35 | 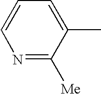 | 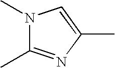 | Br | H | 382/384[M+H]⁺, APCI |
| Example 36 | 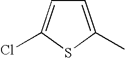 | 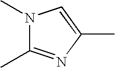 | CN | H | 354/356[M+H]⁺, APCI |

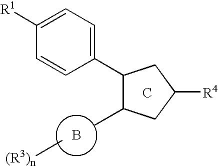

| Example | (R³)ₙ-Ring B | Ring C | R² | R⁴ | MS |
|---|---|---|---|---|---|
| Example 37 | 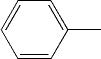 | 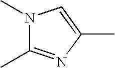 | CN | COOEt | 318[M+H]⁺, APCI |
| Example 38 | 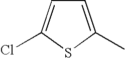 | 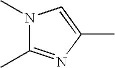 | CONH₂ | CF₃ | 372/374[M+H]⁺, APCI |
| Example 39 | 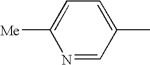 | 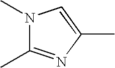 | CONH₂ | CF₃ | 347[M+H]⁺, APCI |

Example 40

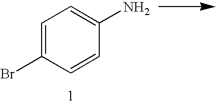

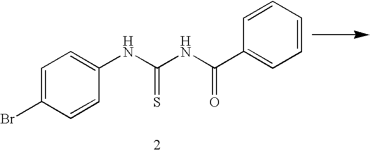

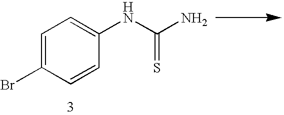

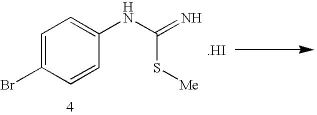

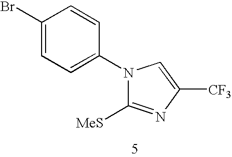

(1) To a THF (200 ml) solution containing benzoyl isothiocyanate (26.10 g, 160 mmol) was dropwise added a THF (200 ml) solution containing 4-bromoaniline at room temperature over 12 minutes under argon atmosphere. After stirring for an hour at the same temperature, to the reaction solution was added NH-silica gel (100 ml) and the mixture was stirred for 20 minutes. Further, NH-silica gel (100 ml) was added thereto and stirred for 20 minutes. NH-silica gel was collected by filtration and washed with ethyl acetate. The filtrate and the washing solution were combined and then concentrated under reduced pressure. The obtained residue was triturated with ethyl acetate-hexane to give Compound 2 (40.95 g, 100%) as powders.
MS:335,337 [M+H]⁺, APCI
(2) Compound 2 (37.94 g, 113 mmol) was suspended in 10% sodium hydroxide (400 ml) and refluxed under heating for 30 minutes. After cooling by allowing to stand, the reaction mixture was diluted with water. The precipitated solid was collected by filtration, washed with water and dried to give Compound 3 (15.70 g, 60%) as powders.
MS:231,233 [M+H]⁺, APCI
(3) To an acetone (300 ml) suspension containing Compound 3 (18.54 g, 80.2 mmol) was added dropwise iodomethane (12.89 g, 90.8 mmol) at room temperature over 10 minutes. After stirring for 23.5 hours at the same temperature, to the reaction solution was added diethyl ether (300 ml). The precipitated solid was collected by filtration, washed with diethyl ether and dried to give Compound 4 (27.96 g, 93%) as powders.
MS:245,247 [M+H]⁺, APCI
(4) An isopropyl alcohol (600 ml) suspension containing Compound 4 (15.75 g, 42.2 mmol), 3-bromo-1,1,1-trifluoroacetone (6.6 ml, 63.6 mmol) and sodium hydrogen carbonate (7.74 g, 92.1 mmol) was refluxed under heating for 5.5 hours. The reaction mixture was concentrated under reduced pressure, the obtained residue was diluted with water and the mixture was extracted with ethyl acetate. The organic layer was washed with brine, dried over anhydrous sodium sulfate, and then concentrated under reduced pressure. The obtained residue was suspended in toluene (300 ml), para-toluenesulfonic acid monohydrate (413 mg) was added thereto, and the mixture was refluxed under heating for 2 hours. The reaction mixture was poured into an aqueous saturated sodium bicarbonate solution and extracted with ethyl acetate. The organic layer was washed with brine, dried over anhydrous sodium sulfate, and then concentrated under reduced pressure. The obtained residue was purified by silica gel column chromatography (hexane:ethyl acetate=15:1->10:1) to give Compound 5 (7.88 g, 55%) as powders.
MS: 337/339 [M+H]⁺, APCI

### EXAMPLE 41

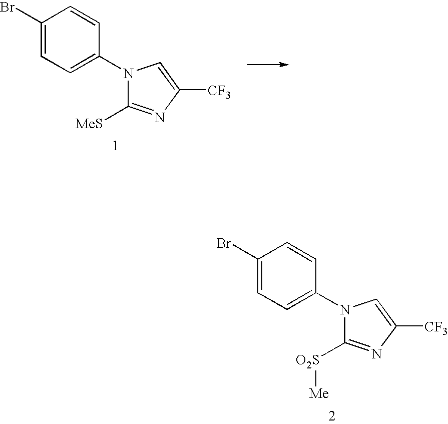

To a methylene chloride (100 ml) solution containing Compound 1 (5.69 g, 16.9 mmol) were added meta-chloroperoxybenzoic acid (4.55 g, 19.8 mmol) at room temperature. After stirring for 20 minutes at the same temperature, meta-chloroperoxybenzoic acid (4.59 g, 19.9 mmol) was added thereto and the mixture was further stirred for 18.5 hours. To the reaction solution was added a 10% aqueous sodium sulfite solution and the reaction mixture was stirred for 40 minutes and extracted with chloroform. The organic layer was washed with brine, dried over anhydrous sodium sulfate, and then concentrated under reduced pressure. The obtained residue was purified by silica gel column chromatography (hexane:ethyl acetate=6:1) to give Compound 2 (6.48 g, 100%) as powders.

MS: 369/371 [M+H]⁺, APCI

### EXAMPLES 42-49

The following compounds were prepared in the same manner as in the above mentioned Examples.

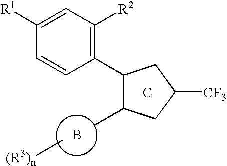

| Example | (R³)ₙ-Ring B | Ring C | R¹ | R² | MS |
|---|---|---|---|---|---|
| Example 42 | 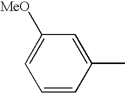 | 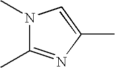 | CONH₂ | H | 362[M+H]⁺, APCI |
| Example 43 | 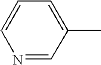 | 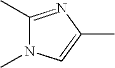 | CONH₂ | H | 333[M+H]⁺, APCI |
| Example 44 | 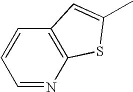 | 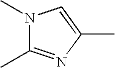 | CONH₂ | H | 389[M+H]⁺, APCI |
| Example 45 | 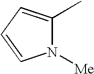 | 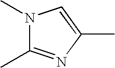 | CONH₂ | H | 335[M+H]⁺, APCI |
| Example 46 | 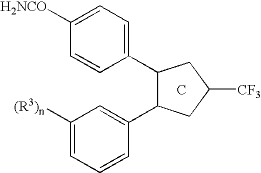 | 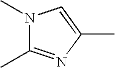 | CONH₂ | H | 347[M+H]⁺, APCI |
| Example 47 | 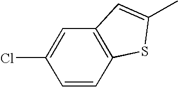 | 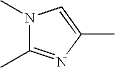 | CONH₂ | H | 422/424[M+H]⁺, APCI |
| Example 48 | 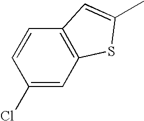 | 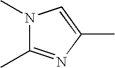 | CONH₂ | H | 422/424[M+H]⁺, APCI |
| Example 49 | 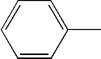 | 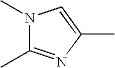 | CONH₂ | Me | 346[M+H]⁺, APCI |

Example 50

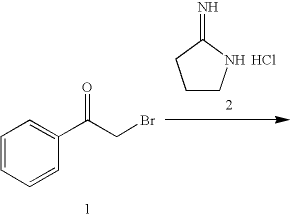

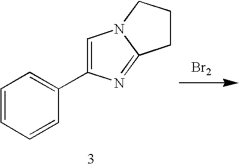

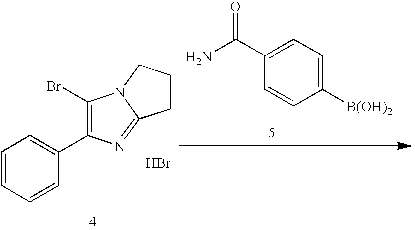

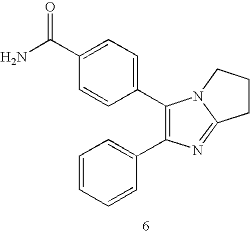

(1) A DMA (35 ml) suspension containing Compound 1 (5.57 g, 28 mmol), Compound 2 (10 g, 83 mmol) and sodium carbonate (12 g, 113 mmol) was stirred at 80° C. for 15 hours under argon atmosphere. After cooling by allowing to stand, the reaction mixture was diluted with ethyl acetate and washed with water. The organic layer was dried over anhydrous sodium sulfate, and then, concentrated under reduced pressure. The residue was purified by NH silica gel column chromatography (hexane:ethyl acetate=3:2) to give Compound 3 (2.02 g, 11 mmol, 39%) as a solid.

MS: 185[M+H]⁺, APCI (MeOH)

(2) To a dichloromethane (50 ml) solution containing Compound 3 (1.13 g, 6.12 mmol) was added dropwise bromine (0.35 ml, 6.73 mmol) at room temperature over 2 minutes and the mixture was stirred for 20 minutes. The solvent was removed, and the residue was triturated with diethyl ether to give Compound 4 (1.93 g, 5.61 mmol, 92%) as a solid.

MS:263/265 [M+H]⁺, APCI(MeOH)

(3) A dimethoxyethane (3 ml) suspension containing Compound 4 (10 mg, 0.29 mmol), Compound 5 (94 mg, 0.57 mmol), dichlorobis(triphenylphosphine)palladium(II)(27 mg, 0.04 mmol) and a 2M aqueous sodium carbonate solution (0.57 ml, 1.14 mmol) was refluxed under heating for 1 hour and 30 minutes under argon atmosphere. After cooling by allowing to stand, the reaction mixture was diluted with water and extracted with ethyl acetate. The organic layer was dried over anhydrous sodium sulfate, and then concentrated under reduced pressure. The residue was purified by NH silica gel column chromatography (chloroform:methanol=19:1) to give Compound 6 (41 mg, 0.135 mmol, 46%) as powders.

MS:304[M+H]⁺, APCI(MeOH)

### EXAMPLE 51, 52

The following compounds were prepared by reacting and treating in the same manner as in Example 50.

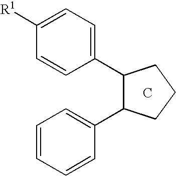

| Example | Ring C | R¹ | MS |
|---|---|---|---|
| Example 51 | 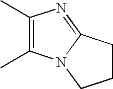 | CONH₂ | 304[M+H]⁺, APCI |
| Example 52 | 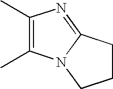 | CN | 286[M+H]⁺, APCI |

### EXAMPLE 53-55

The following compounds were prepared in the same manner as in the above mentioned Examples.

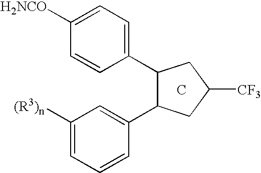

| Example | Ring C | R³ | n | MS |
|---|---|---|---|---|
| Example 53 | 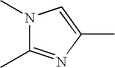 | F | 1 | 350[M+H]⁺, APCI |
| Example 54 | 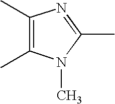 | — | 0 | 346[M+H]⁺, APCI |
| Example 55 | 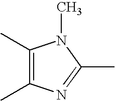 | — | 0 | 346[M+H]⁺, APCI |

Example 56

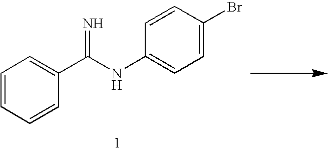

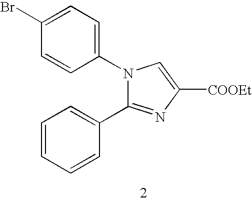

An isopropyl alcohol (250 ml) suspension containing Compound 1 (5.61 g, 20.4 mmol), ethyl 3-bromopyruvate (5.0 ml, 31.9 mmol) and sodium hydrogen carbonate (3.50 g, 41.7 mmol) was refluxed under heating for 15 hours. The reaction mixture was concentrated under reduced pressure, the obtained residue was diluted with water and the mixture was extracted with ethyl acetate. The organic layer was washed with brine, dried over anhydrous sodium sulfate, and then concentrated under reduced pressure. The obtained residue was purified by NH silica gel column chromatography (hexane:ethyl acetate=7:1->3:1) to give Compound 2 (5.00 g, 66%) as powders.

MS:371/373 [M+H]⁺, APCI

### EXAMPLES 57-84

The following compounds were prepared in the same manner as in the above mentioned Examples.

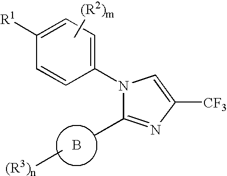

| Example | (R³)ₙ-Ring B | (R²)ₘ | MS |
|---|---|---|---|
| Example 57 | 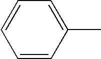 | 3-Me | 381/383[M+H]⁺, APCI |
| Example 58 | 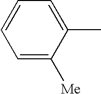 | H | 381/383[M+H]⁺, APCI |
| Example 59 | 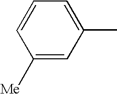 | H | 381/383[M+H]⁺, APCI |
| Example 60 | 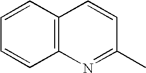 | H | 418/420[M+H]⁺, APCI |
| Example 61 | 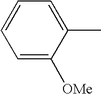 | H | 397/399[M+H]⁺, APCI |
| Example 62 | 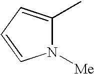 | H | 370/372[M+H]⁺, APCI |
| Example 63 | 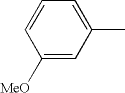 | H | 397/399[M+H]⁺, APCI |
| Example 64 | 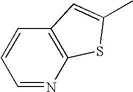 | H | 424/426[M+H]⁺, APCI |
| Example 65 | 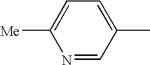 | H | 382/384[M+H]⁺, APCI |
| Example 66 | 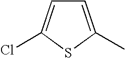 | H | 407/409[M+H]⁺, APCI |
| Example 67 | 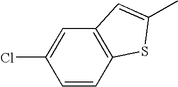 | H | 457/459[M+H]⁺, APCI |
| Example 68 | 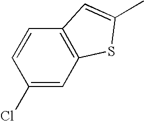 | H | 457/459[M+H]⁺, APCI |
| Example 69 | 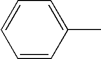 | 2-Me | 381/383[M+H]⁺, APCI |

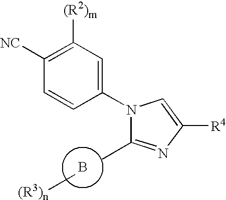

| Example | (R³)ₙ-Ring B | (R²)ₘ | R⁴ | MS |
|---|---|---|---|---|
| Example 70 | 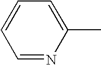 | H | CF₃ | 315[M + H]⁺, APCI |
| Example 71 | 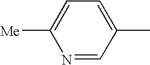 | H | CF₃ | 329[M + H]⁺, APCI |
| Example 72 | 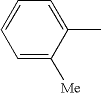 | H | CF₃ | 328[M + H]⁺, APCI |
| Example 73 | 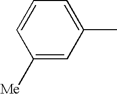 | H | CF₃ | 328[M + H]⁺, APCI |
| Example 74 | 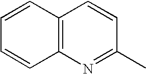 | H | CF₃ | 365[M + H]⁺, APCI |
| Example 75 | 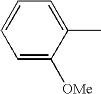 | H | CF₃ | 344[M + H]⁺, APCI |

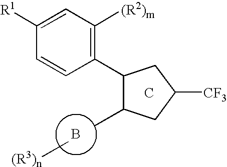

| Example | (R³)ₙ-Ring B | Ring C | R¹ | (R²)ₘ | MS |
|---|---|---|---|---|---|
| Example 76 | 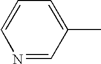 | 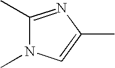 | CN | H | 315[M + H]⁺, APCI |
| Examp1e 77 | 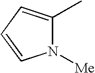 | 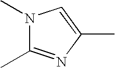 | CN | H | 317[M + H]⁺, APCI |
| Example 78 | 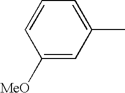 | 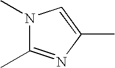 | CN | H | 344[M + H]⁺, APCI |
| Example 79 | 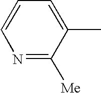 | 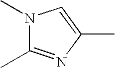 | CN | H | 329[M + H]⁺, APCI |
| Example 80 | 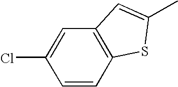 | 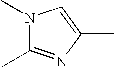 | CN | H | 404/406[M + H]⁺, APCI |
| Example 81 | 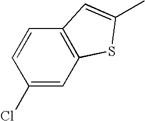 | 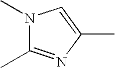 | CN | H | 404/406[M + H]⁺, APCI |
| Example 82 | 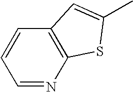 | 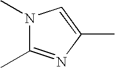 | CN | H | 371[M + H]⁺, APCI |
| Example 83 | 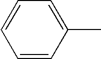 | 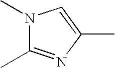 | CN | H | 328[M + H]⁺, APCI |
| Example 84 | 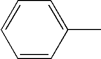 | 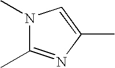 | COOH | H | 331[M + H]⁺, ESI |

Example 85

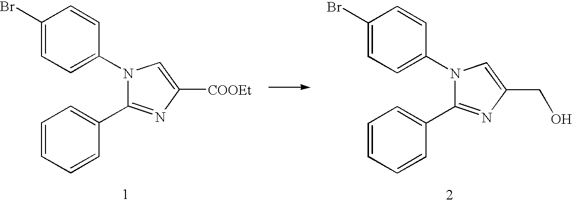

To a THF (50 ml) suspension containing lithium aluminum hydride (280 mg, 7.38 mmol) was added dropwise a THF (50 ml) solution containing Compound 1 (2.46 g, 6.63 mmol) under argon atmosphere, and the mixture was stirred at room temperature for 8 hours and a half. To the reaction solution were added water (0.28 ml) and 5N sodium hydroxide solution (0.28 ml) under ice-cooling and water (0.84 ml) was further added thereto and the mixture was stirred at room temperature for 17 hours. After filtering out insoluble matters and washing with THF, the filtrate and the washing solution were combined and then concentrated under reduced pressure. The residue was triturated with ethyl acetate-diethyl ether to give Compound 2 (1.20 g, 55%) as powders.

MS:329/331 [M+H]⁺, APCI

### EXAMPLE 86

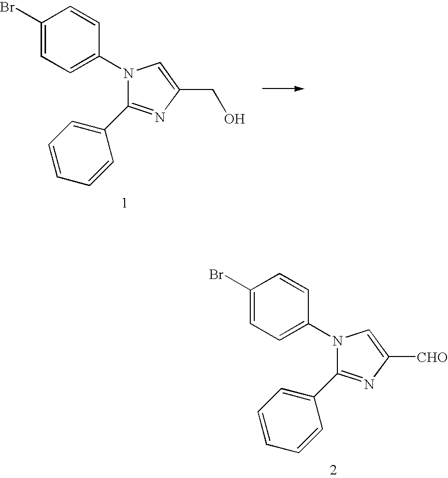

To a chloroform (35 ml) solution containing Compound 1 (330 mg, 11.0 mmol) was added manganese dioxide (85%, 536 mg, 5.2 mmol) at room temperature and the mixture was refluxed under heating for 17 hours and a half. After cooling the reaction mixture, insoluble matters were filtered through Celite and washed with chloroform. The filtrate and the washing solution were combined and then concentrated under reduced pressure to give Compound 2 (334 mg, 100%) as a solid.

MS:327/329 [M+H]⁺, APCI

### EXAMPLE 87

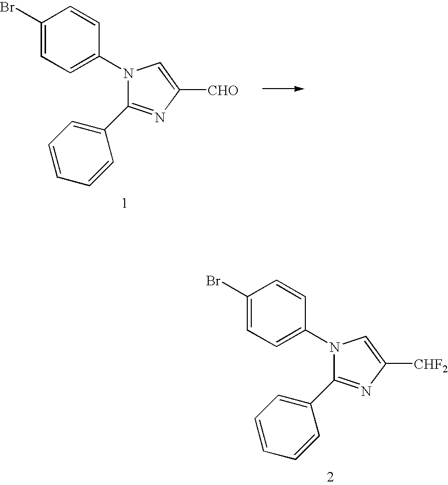

To a methylene chloride (5 ml) solution containing Compound 1 (99 mg, 0.302 mmol) was added diethylaminosulfur trifluoride (0.15 ml, 1.14 mmol) under argon atmosphere and the mixture was stirred at room temperature for 14 hours. The reaction mixture was poured into a cooled and saturated sodium bicarbonate solution and extracted with chloroform. The organic layer was washed with brine, dried over anhydrous sodium sulfate, and then concentrated under reduced pressure. The residue was purified by silica gel column chromatography (hexane:ethyl acetate=85:15->50:50) to give Compound 2 (78 mg, 74%) as an oil.

MS: 349/351 [M+H]⁺, APCI

### EXAMPLE 88

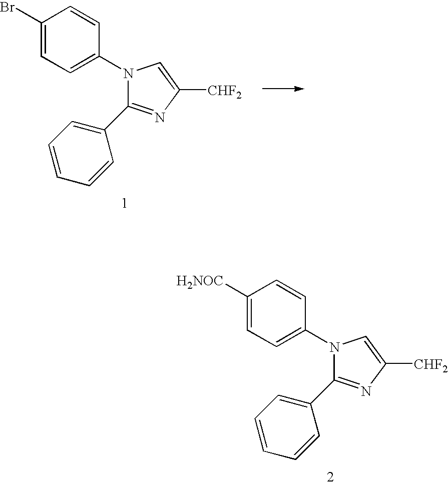

Compound 2 was prepared using Compound 1 by reacting and treating in the same manner as in Example 1, (2) and (3).

MS:314 [M+H]⁺, APCI

### EXAMPLES 89-103

The following compounds were prepared in the same manner as in the above mentioned Examples.

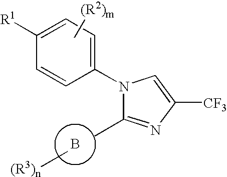

| Example | (R³)ₙ-Ring B | R¹ | (R²)ₘ | MS |
|---|---|---|---|---|
| Example 89 | 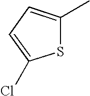 | COOH | H | 371/373[M − H]⁻, ESI |
| Example 90 | 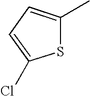 | CONH₂ | 2-Me | 366/368[M + H]⁺, APCI |
| Example 91 | 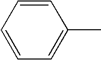 | 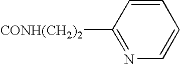 | H | 451[M + H]⁺, ESI |
| Example 92 | 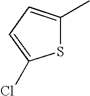 | CONHCH₂CH(OH)CH₂OH (S) | H | 446[M + H]⁺, ESI |
| Example 93 | 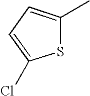 | CONHCH₂CH(OH)CH₃ (R) | H | 430[M + H]⁺, ESI |
| Example 94 | 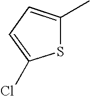 | CONH(CH₂)₂OH | H | 416[M + H]⁺, ESI |
| Example 95 | 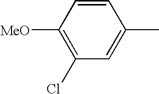 | CONH₂ | H | 396/398[M + H]⁺, APCI |
| Example 96 | 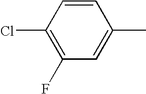 | CONH₂ | H | 384[M + H]⁺, ESI |
| Example 97 | 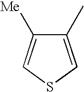 | CONH₂ | H | 352[M + H]⁺, ESI |
| Example 98 | 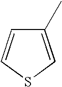 | CONH₂ | H | 338[M + H]⁺, APCI |
| Example 99 | 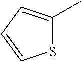 | CONH₂ | H | 338[M + H]⁺, APCI |
| Example 100 | 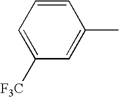 | CONH₂ | H | 400[M + H]⁺, APCI |
| Example 101 | 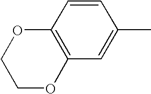 | CONH₂ | H | 390[M + H]⁺, ESI |
| Example 102 | 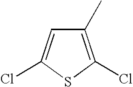 | CONH₂ | H | 406[M + H]⁺, ESI |
| Example 103 | 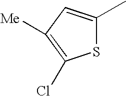 | CONH₂ | H | 386[M + H]⁺, ESI |

Example 104

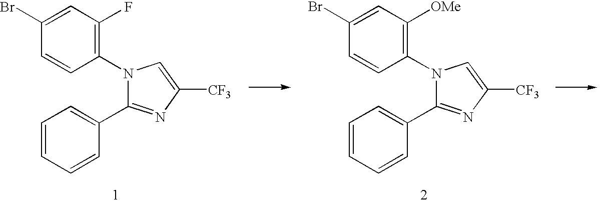

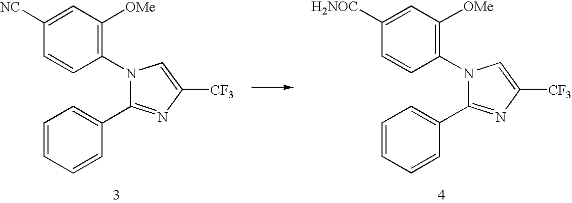

(1) Methanol (2 ml) was added dropwise to sodium hydride (60% purity, 63 mg, 1.56 mmol) at room temperature, the mixture was stirred for 10 minutes and thereto was dropwise added a solution of Compound 1 (382 mg, 0.992 mmol) in methanol (3 ml). The mixture was stirred at room temperature for 3 hours and a half, and further refluxed under heating for 33 hours. The reaction solution was cooled, water was added thereto and the mixture was extracted with ethyl acetate. The organic layer was washed with brine, dried over anhydrous sodium sulfate and concentrated under reduced pressure. The residue was purified by silica gel column chromatography (hexane:ethyl acetate 95:5->80:20) to give Compound 2 (245 mg, 62%) as powders.
MS: 397/399 [M+H]⁺, APCI
(2) A mixture of Compound 2 (243 mg, 0.612 mmol), potassium hexacyanoferrate (II) trihydrate (129 mg, 0.305 mmol), dichloro(diphenylphosphinoferrocene)palladium (15 mg, 0.018 mmol) and sodium carbonate (65 mg, 0.613 mmol) in dimethylacetamide (3 ml) was stirred at 120° C. for 2 hours. The reaction solution was cooled, and thereto were added water and ethyl acetate. Insoluble matters were filtered through Celite and washed with ethyl acetate. The filtrate and the washing solution were combined and extracted with ethyl acetate. The organic layer was washed with water and brine, dried over anhydrous sodium sulfate and concentrated under reduced pressure. The residue was purified by silica gel column chromatography (hexane:ethyl acetate 90:10->70:30) to give Compound 3 (164 mg, 78%) as powders.
MS: 344 [M+H]⁺, APCI
(3) Compound 4 was prepared by reacting and treating in the same manner as in example 1(3) using Compound 3.
MS: 362 [M+H]⁺, APCI

### EXAMPLE 105

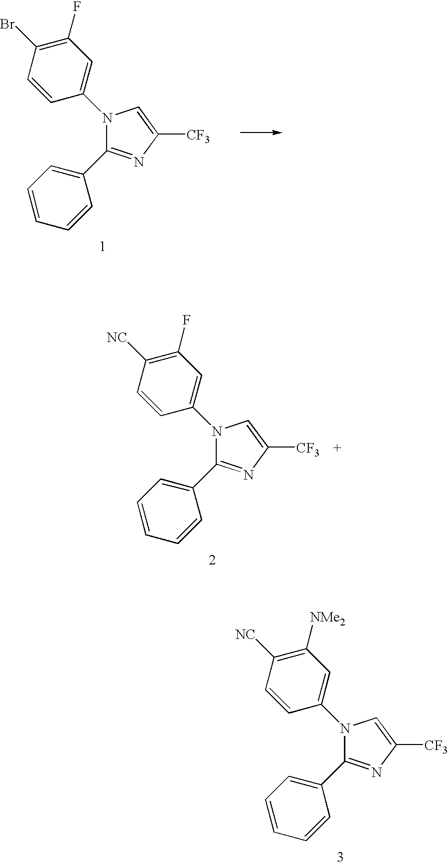

A suspension of Compound 1 (1.38 g, 3.59 mmol), zinc cyanide (257 mg, 2.19 mmol) and tetrakis(triphenylphosphine)palladium (512 mg, 0.443 mmol) in DMF (35 ml) was stirred at 90° C. for 50 hours under argon atmosphere. The reaction solution was cooled, and thereto were added water and ethyl acetate. Insoluble matters were filtered through Celite and washed with ethyl acetate. The filtrate and the washing solution were combined and extracted with ethyl acetate. The organic layer was washed with water and brine, dried over anhydrous sodium sulfate and concentrated under reduced pressure. The residue was purified by silica gel column chromatography (hexane:ethyl acetate=10:1) to give Compound 2 (198 mg, 17%) and Compound 3 (43 mg, 3%), as a solid respectively.

Compound 2

MS: 332 [M+H]⁺, APCI

Compound 3

MS: 357 [M+H]⁺, APCI

### EXAMPLE 106

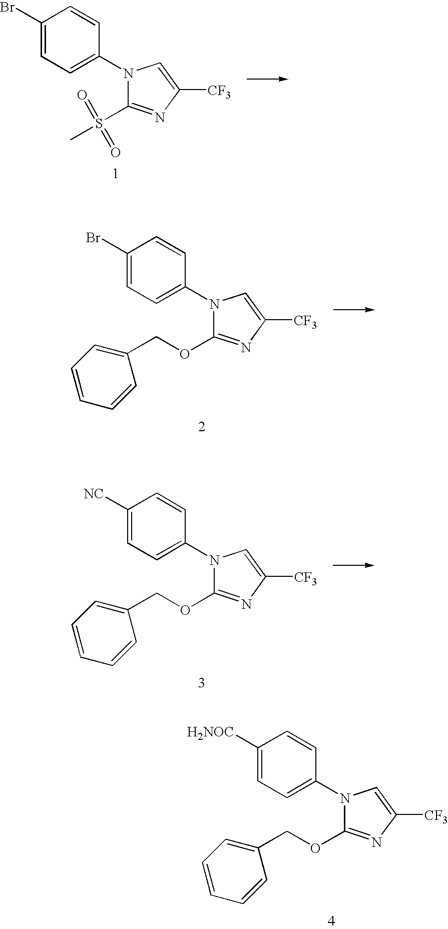 (1) To a solution of benzyl alcohol (13.2 ml, 0.127 mol) in DMF (400 ml) was added sodium hydride (60% purity, 7.30 g, 0.183 mol) at room temperature and the mixture was stirred at 60° C. for 10 minutes. The reaction solution was ice-cooled and thereto was dropwise added a solution of Compound 1 (33.7 g, 0.0913 mol) in DMF (100 ml). The reaction solution was stirred at room temperature for 3 days and the solvent was removed under reduced pressure. To the residue was added diethyl ether and the mixture was washed with a 10% aqueous citric acid solution and brine. The mixture was dried over Chem Elut® and concentrated under reduced pressure. The residue was purified by silica gel column chromatography (hexane:ethyl acetate=100:0->90:10) to give Compound 2 (42.4 g, 84%) as an oil.
MS: 397/399 [M+H]⁺, APCI
(2) Compound 3 was prepared by reacting and treating in the same manner as in example 1(2) using Compound 2.
MS: 344 [M+H]⁺, APCI
(3) Compound 4 was prepared by reacting and treating in the same manner as in example 1(3) using Compound 3.
MS: 362 [M+H]⁺, APCI

### EXAMPLE 107

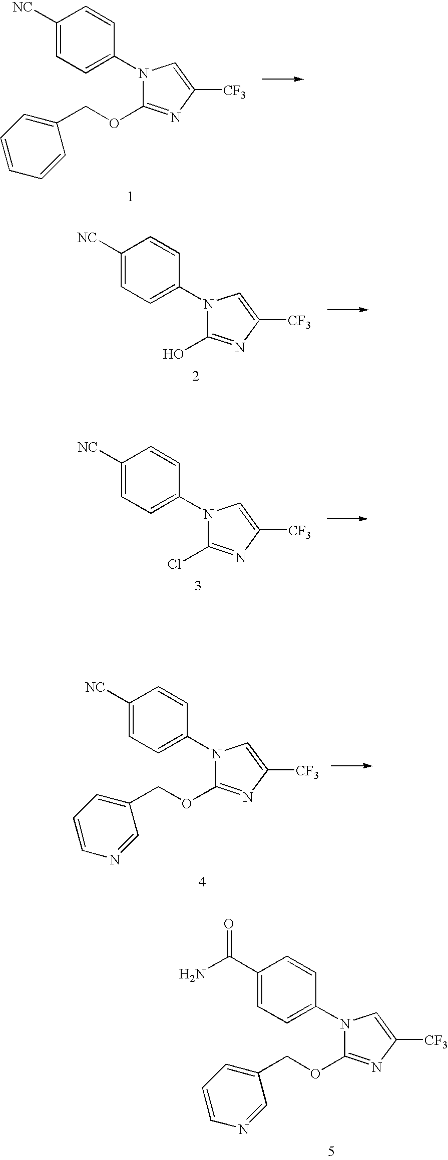 (1) A suspension of Compound 1 (34.0 g, 99.0 mmol) and 10% Pd—C (34 g) in methanol (500 ml) was stirred at room temperature for 2 hours under hydrogen atmosphere. Insoluble matters were removed by filtration, the filtrate was concentrated and the residue was triturated with ethyl acetate-diisopropyl ether to give Compound 2 (15.0 g, 60%) as powders.
MS: 252 [M−H]⁻, ESI
(2) A mixture containing Compound 2 (15.0 g, 55.1 mmol) and phosphorous oxychloride (150 ml) was refluxed under heating for 6 hours. The reaction solution was cooled, concentrated under redued pressure and ethyl acetate was added to the residue. The mixture was washed with brine, dried over anhydrous magnesium sulfate and concentrated under reduced pressure. The residue was purified by silica gel column chromatography (hexane:ethyl acetate=100:0->75:25) to give Compound 3 (13.4 g, 83%) as powders.
MS: 272/274 [M+H]⁺, APCI
(3) To a solution of Compound 3 (100 mg, 0.368 mmol) and 3-pyridinemethanol (60 mg, 0.550 mmol) in THF (3 ml) was added sodium hydride (60% purity, 18 mg, 0.45 mmol), the mixture was stirred at room temperature for 2 days and further stirred at 60° C. for 2 hours. The reaction solution was filtered by Chem Elut® and Bond Elut NH2®, and washed with ethyl acetate. The filtrate and the washing solution were combined and concentrated under reduced pressure. The residue was purified by silica gel column chromatography (chloroform:methanol=95:5) to give Compound 4 (58.8 mg, 46%) as powders.
MS: 345 [M+H]⁺, APCI
(4) Compound 5 Was prepared by reacting and treating in the same manner as in example 1(3) using Compound 4.
MS: 363 [M+H]⁺, APCI

### EXAMPLE 108

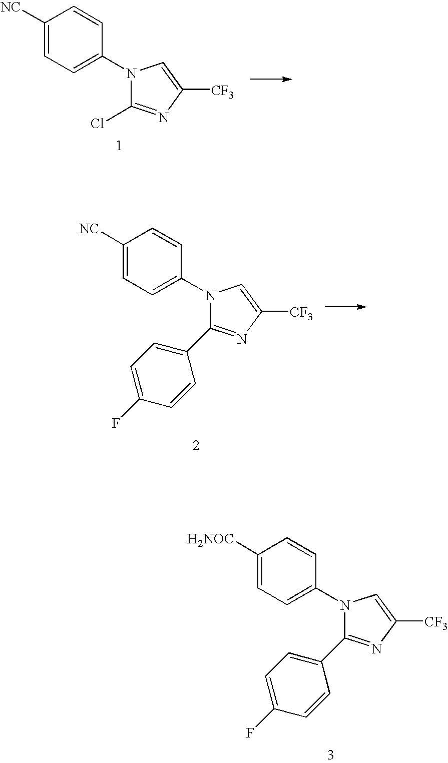 (1) A mixture of Compound 1 (60 mg, 0.221 mmol),
dichlorobis(triphenylphosphine)palladium (16 mg, 0.023 mmol) and 4-fluorophenylboric acid (34 mg, 0.243 mmol) in a 2M aqueous sodium carbonate solution (0.44 ml) and acetonitrile (0.44 ml) was stirred at 110° C. for 4 hours under argon atmosphere. The reaction solution was cooled and thereto was added ethyl acetate. The mixture was washed with water, dried by Chem Elut® and concentrated under reduced pressure. The residue was purified by silica gel column chromatography (hexane:ethyl acetate=95:5->75:25) to give Compound 2 (62.3 mg, 85%) as powders.
MS: 332 [M+H]⁺, APCI
(2) Compound 3 was prepared by reacting and treating in the same manner as in example 1(3) using Compound 2.
MS: 350 [M+H]⁺, APCI

### EXAMPLE 109

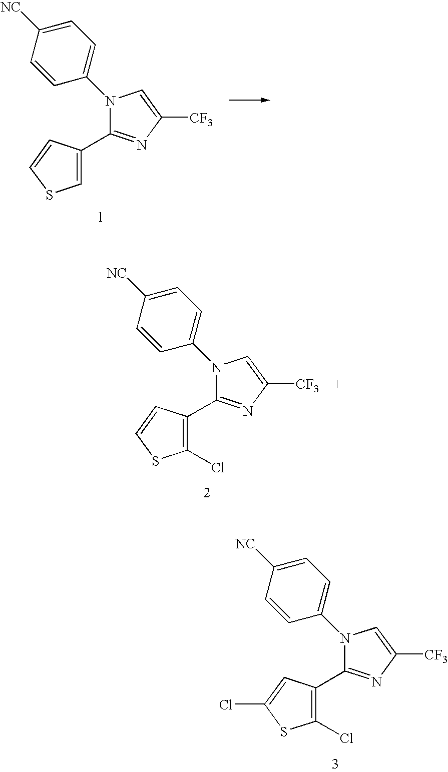

A solution of Compound 1 (330 mg, 1.03 mmol) and N-chlorosuccinimide (304 mg, 2.28 mmol) in acetic acid (5 ml) was stirred at room temperature for 2 days. The reaction solution was poured into a cooled saturated sodium hydrogen carbonate solution and extracted with diethyl ether. The organic layer was washed with brine, dried over anhydrous magnesium sulfate and concentrated under reduced pressure.

The residue was purified by silica gel column chromatography (hexane:ethyl acetate=100:0->75:25) to give Compound 2 (146 mg, 40%) and Compound 3 (131 mg, 33%), as powders respectively.

Compound 2

MS: 354/356 [M+H]⁺, APCI

Compound 3

MS: 388/390 [M+H]⁺, APCI

### EXAMPLE 110

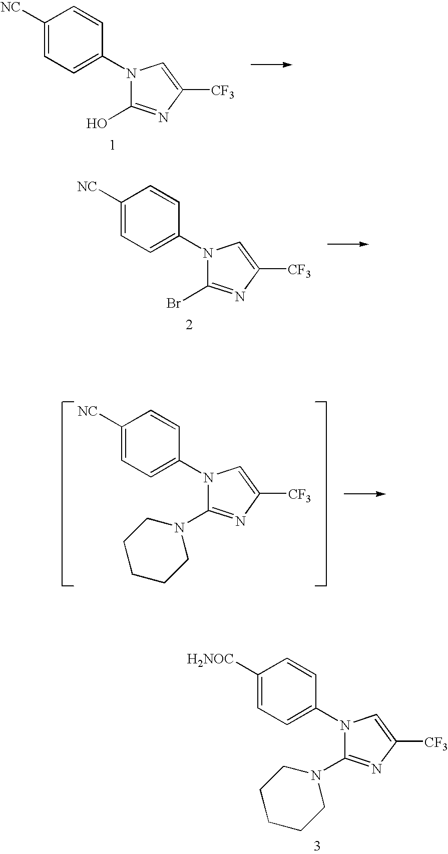 (1) A mixture containing Compound 1 (1.2 g, 47.4 mmol) and phosphorous oxybromide (30 g, 105 mmol) was stirred at 130° C. for an hour. The reaction solution was cooled, poured into a cooled aqueous potassium carbonate solution and extracted with ethyl acetate. The organic layer was washed with brine, dried over anhydrous magnesium sulfate and concentrated under reduced pressure. The residue was purified by silica gel column chromatography (hexane:ethyl acetate=100:0->75:25) to give Compound 2 (994 mg, 66%) as powders.
MS: 316/318 [M+H]⁺, APCI
(2) A solution of Compound 2 (100 mg, 0.316 mmol), piperidine (33 mg, 0.388 mmol) and potassium carbonate (44 mg, 0.318 mmol) in DMF (2 ml) was stirred at 60° C. for a day, the mixture was stirred at 90° C. for a day and further stirred at 120° C. for a day. The reaction solution was cooled, thereto was added ethyl acetate. The mixture was washed with brine, dried over anhydrous magnesium sulfate and concentrated under reduced pressure. To the residue were added potassium hydroxide powder (160 mg, 2.85 mmol) and t-butanol (10 ml) and the mixture was stirred at 80° C. for 30 minutes. The reaction solution was cooled, concentrated and thereto was added ethyl acetate. The mixture was washed with brine, dried by Chem Elut® and concentrated under reduced pressure. The residue was purified by silica gel column chromatography (chloroform:methanol=100:0->90:10) to give Compound 3 (27 mg, 25%) as powders.
MS: 339 [M+H]⁺, APCI

### EXAMPLE 111-148

The following compounds were prepared in the same manner as in the above mentioned Examples.

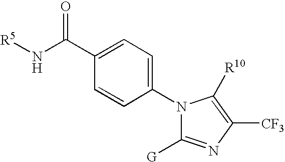

| Example | R⁵ | G | R¹⁰ | MS |
|---|---|---|---|---|
| Example 111 | H | iPrO— | H | 314[M + H]⁺, APCI |
| Example 112 | H | 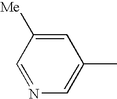 | H | 347[M + H]⁺, APCI |
| Example 113 | 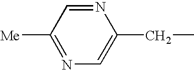 | 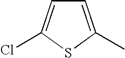 | H | 478[M + H]⁺, ESI |
| Example 114 | H₂NCOCH₂— | 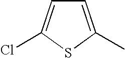 | H | 429[M + H]⁺, ESI |
| Example 115 | 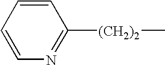 | 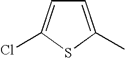 | H | 477[M + H]⁺, ESI |
| Example 116 | H | 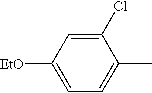 | H | 410[M + H]⁺, ESI |
| Example 117 | H | 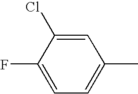 | H | 384[M + H]⁺, ESI |
| Example 118 | H | 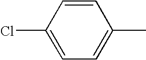 | H | 366[M + H]⁺, ESI |
| Example 119 | H | 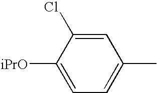 | H | 424[M + H]⁺, ESI |
| Example 120 | H | 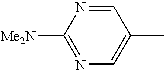 | H | 377[M + H]⁺, APCI |
| Example 121 | H | 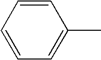 | Me | 346[M + H]⁺, APCI |
| Example 122 | H | 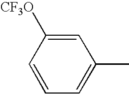 | H | 416[M + H]⁺, APCI |

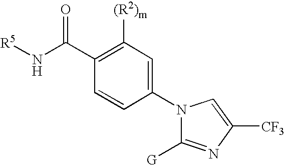

| Example | R⁵ | G | (R²)ₘ | MS |
|---|---|---|---|---|
| Example 123 | H | 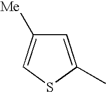 | — | 352[M + H]⁺, ESI |
| Example 124 | H | 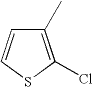 | — | 372[M + H]⁺, ESI |
| Example 125 | H | 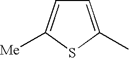 | — | 352[M + H]⁺, ESI |
| Example 126 | H | 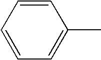 | OMe | 362[M + H]⁺, APCI |
| Example 127 | H | 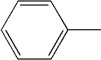 | NMe₂ | 375[M + H]⁺, APCI |
| Example 128 | H | 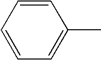 | F | 350[M + H]⁺, APCI |
| Example 129 | H | 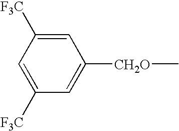 | — | 498[M + H]⁺, ESI |
| Example 130 | H | 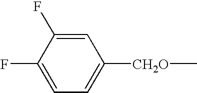 | — | 398[M + H]⁺, ESI |
| Example 131 | H | 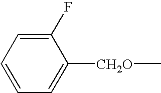 | — | 380[M + H]⁺, ESI |
| Example 132 | H | 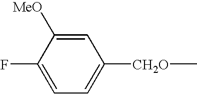 | — | 410[M + H]⁺, ESI |
| Example 133 | H | 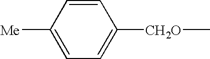 | — | 376[M + H]⁺, ESI |

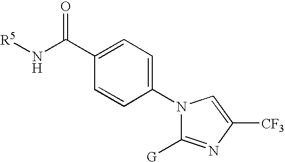

| Example | R⁵ | G | MS |
|---|---|---|---|
| Example 134 | H | 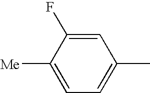 | 364[M + H]⁺, APCI |
| Example 135 | H | 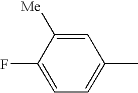 | 364[M + H]⁺, APCI |
| Example 136 | H | 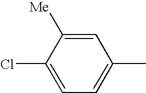 | 380[M + H]⁺, APCI |
| Example 137 | H | 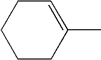 | 336[M + H]⁺, APCI |
| Example 138 | H | 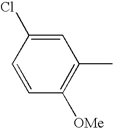 | 396/398[M + H]⁺, APCI |
| Example 139 | H | 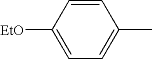 | 376[M + H]⁺, APCI |
| Example 140 | H | 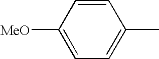 | 362[M + H]⁺, APCI |
| Example 141 | H | 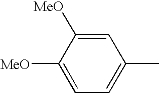 | 392[M + H]⁺, APCI |
| Example 142 | H | 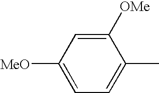 | 392[M + H]⁺, APCI |

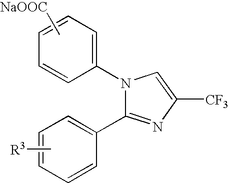

| | position of | | |
|---|---|---|---|
| Example | COONa | R³ | MS |
| Example 143 | 4-position | 3-F | 349[M − Na]⁻, ESI |
| Example 144 | 3-position | 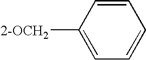 | 437[M − Na]⁻, ESI |

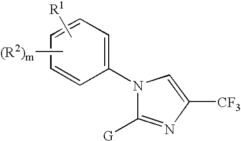

| Example | R¹ | (R²)ₘ | G | MS |
|---|---|---|---|---|
| Example 145 | 3-CONH₂ | — | 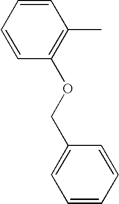 | 438[M + H]⁺, APCI |
| Example 146 | 4-CONH₂ | 2-F | 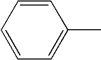 | 350[M + H]⁺, APCI |
| Example 147 | 4-CONH₂ | 2,6-diMe | 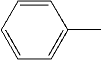 | 360[M + H]⁺, APCI |
| Example 148 | 4-CONH₂ | 3-Me | 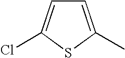 | 386/388[M + H]⁺, APCI |

Example 149

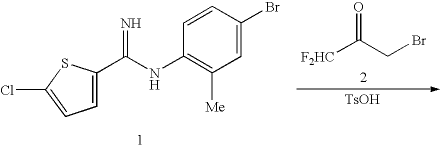

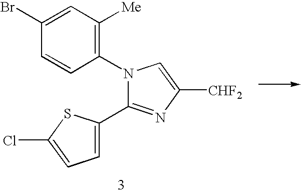

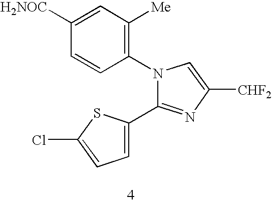

(1) Preparation of 1-Bromo-3′,3′-difluoroacetone (Compound 2) To a mixture of ethyl difluoroacetate (1.6 ml, 15.2 mmol) and dibromomethane (1.93 ml, 27.5 mmol) in THF (60 ml) was added dropwise methyllithium (1.04 M solution in diethyl ether; 26.4 ml, 27.5 mmol) over 15 min at −78° C., and the mixture was stirred for 30 minutes at the same temperature. To the mixture was added saturated aqueous ammonium chloride solution (10 ml) at the same temperature, then the mixture was warmed to room temperature, diluted with water and extracted with diethyl ether. The organic layer was dried over magnesium sulfate, and concentrated under reduced pressure to give crude Compound 2 (4.17 g). The crude product was used without purification.
(2) A mixture of Compound 1 (209 mg, 0.634 mmol), Compound 2 (877 mg) and sodium bicarbonate (107 mg, 1.27 mmol) in isopropanol (10 ml) was refluxed for 45 minutes. After cooling, the mixture was diluted with water and extracted with chloroform. The organic layer was dried over sodium sulfate and concentrated under reduced pressure. The residue was dissolved in toluene (5 ml) and p-toluenesulfonic acid monohydrate (24 mg, 0.126 mmol) was added thereto, then the mixture was refluxed for 45 minutes. After cooling, the mixture was diluted with saturated aqueous sodium bicarbonate solution and extracted with ethyl acetate. The organic layer was dried over sodium sulfate, and concentrated under reduced pressure. The residue was purified by silica gel column chromatography (hexane:ethyl acetate=90:10->80:20) to give Compound 3 (219 mg, 86%) as a syrup.
MS: 403/405 [M+H]⁺, APCI
(3) Compound 4 was prepared by reacting and treating in the same manner as in example 1(2) and (3) using compound 3.
MS: 368/370 [M+H]⁺, APCI

### EXAMPLE 150-154

The following compounds were prepared in the same manner as in the above mentioned Examples.

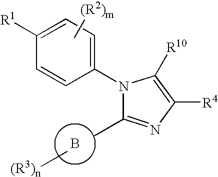

| Example | (R³)ₙ-Ring B | R¹ | (R²)ₘ | R⁴ | R¹⁰ | MS |
|---|---|---|---|---|---|---|
| Example 150 | 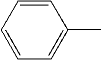 | CONH₂ | 3-Me | CF₃ | Me | 360[M + H]⁺, APCI |
| Example 151 | 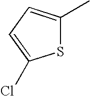 | CONH₂ | H | CHF₂ | H | 354/356[M + H]⁺, APCI |
| Example 152 | 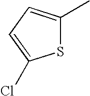 | CONH₂ | 3-Me | CHF₂ | H | 368/370[M + H]⁺, APCI |
| Example 153 | 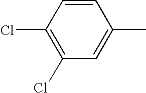 | CONH₂ | H | CF₃ | H | 400/402[M + H]⁺, APCI |
| Example 154 | 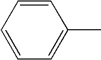 | COOH | 2-Me | CF₃ | H | 345[M − H]⁻, ESI |

The following compounds can be prepared in the same manner as in the above-mentioned Examples.

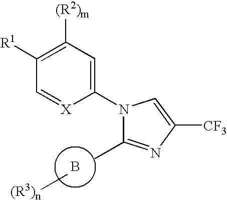

| Compound | (R³)ₙRing B | R¹ | R² | m | X |
|---|---|---|---|---|---|
| (1) | 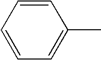 | CONH₂ | — | 0 | N |
| (2) | 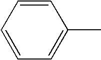 | CONH₂ | Cl | 1 | CH |
| (3) | 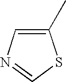 | CONH₂ | — | 0 | CH |
| (4) | 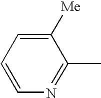 | CONH₂ | — | 0 | CH |
| (5) | 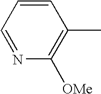 | CONH₂ | — | 0 | CH |
| (6) | 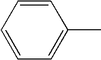 | CONH₂ | — | 0 | N |
| (7) | 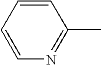 | CONH₂ | — | 0 | CH |
| (8) | 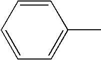 | CONH₂ | CH₃ | 1 | CH |
| (9) | 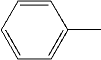 | CONH₂ | F | 1 | CH |
| (10) | 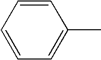 | CONH₂ | Cl | 1 | CH |
| (11) | 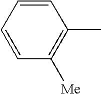 | CONH₂ | — | 0 | CH |
| (12) | 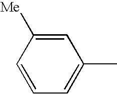 | CONH₂ | — | 0 | CH |
| (13) | 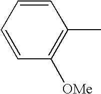 | CONH₂ | — | 0 | CH |
| (14) | 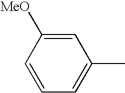 | CONH₂ | — | 0 | CH |
| (15) | 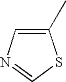 | CONH₂ | — | 0 | CH |
| (16) | 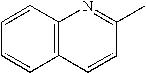 | CONH₂ | — | 0 | CH |
| (17) | 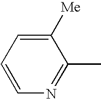 | CONH₂ | — | 0 | CH |
| (18) | 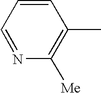 | CONH₂ | — | 0 | CH |
| (19) | 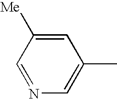 | CONH₂ | — | 0 | CH |
| (20) | 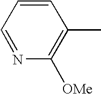 | CONH₂ | — | 0 | CH |
| (21) | 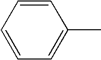 | CONH₂ | — | 0 | N |
| (22) | 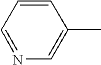 | CONH₂ | — | 0 | CH |
| (23) | 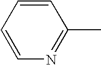 | CONH₂ | — | 0 | CH |
| (24) | 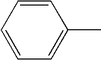 | CONH₂ | CH₃ | 1 | CH |
| (25) | 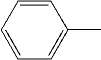 | CONH₂ | Cl | 1 | CH |
| (26) | 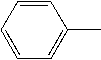 | CONH₂ | F | 1 | CH |
| (27) | 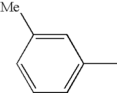 | CONH₂ | — | 0 | CH |
| (28) | 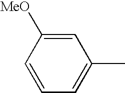 | CONH₂ | — | 0 | CH |
| (29) | 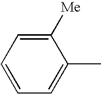 | CONH₂ | — | 0 | CH |
| (30) | 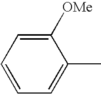 | CONH₂ | — | 0 | CH |
| (31) | 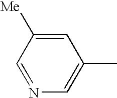 | CONH₂ | — | 0 | CH |
| (32) | 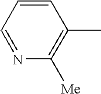 | CONH₂ | — | 0 | CH |
| (33) | 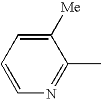 | CONH₂ | — | 0 | CH |
| (34) | 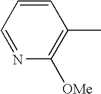 | CONH₂ | — | 0 | CH |
| (35) | 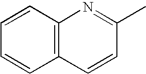 | CONH₂ | — | 0 | CH |

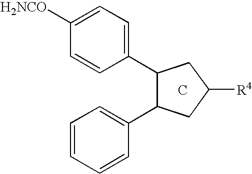

| Compound | Ring C | R⁴ |
|---|---|---|
| (36) | 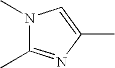 | COONa |
| (37) | 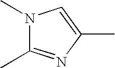 | NHCOO^{t}Bu |
| (38) | 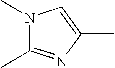 | 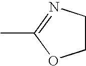 |
| (39) | 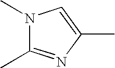 | NH₂ |
| (40) | 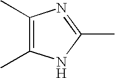 | CH₃ |

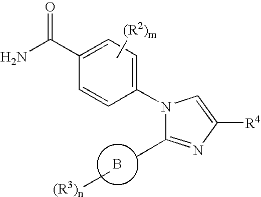

| Compound | (R³)ₙ-Ring B | R² | m | R⁴ |
|---|---|---|---|---|
| (41) | 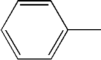 | 2-Me | 1 | CHF₂ |
| (42) | 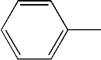 | 3-Me | 1 | CHF₂ |
| (43) | 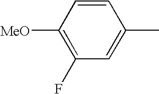 | — | 0 | CF₃ |
| (44) | 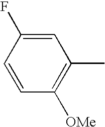 | — | 0 | CF₃ |
| (45) | 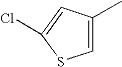 | — | 0 | CF₃ |
| (46) | 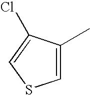 | — | 0 | CF₃ |

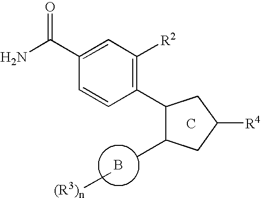

| Compound | (R³)ₙ-Ring B | Ring C | R² | R⁴ |
|---|---|---|---|---|
| (47) | 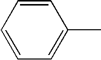 | 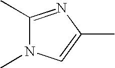 | Me | CF₃ |
| (48) | 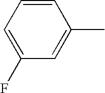 | 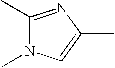 | H | CF₃ |
| (49) | 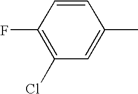 | 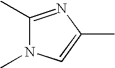 | H | CF₃ |
| (50) | 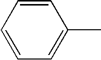 | 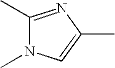 | H | CHF₂ |
| (51) | 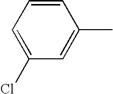 | 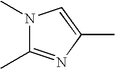 | H | CF₃ |
| (52) | 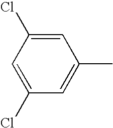 | 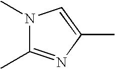 | H | CF₃ |
| (53) | 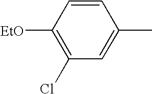 | 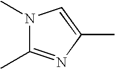 | H | CF₃ |
| (54) | 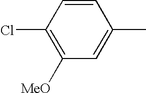 | 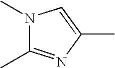 | H | CF₃ |
| (55) | 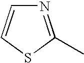 | 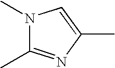 | H | CF₃ |
| (56) | 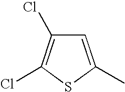 | 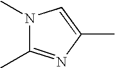 | H | CF₃ |

### Experimental example 1

### Relaxation Effect on Potassium-Induced Contraction of Isolated Rabbit Urinary Bladder

Urinary bladder was isolated from rabbits (body weight: 2.0 kg to 3.5 kg) and immersed in ice-cold Krebs-bicarbonate solution (in mM: 118 NaCl, 4.7 KCl, 2.55 CaCl₂, 1.18 MgSO₄, 1.18 KH₂PO₄, 24.88 NaHCO₃ and 11.1 glucose). The urinary bladder was cut into longitudinal strips (5 mm length, 3-4 mm width) after mucosal layer was removed.

Preparations were mounted in organ baths containing 10 ml of Krebs solution maintained at 37° C. and gassed with 95% O₂/5% CO₂. Accordingly, preparations were stretched with an initial tension of 2.01±1.0 g, and changes in isometric tension were measured by force-displacement transducer. The preparations were pre-contracted by changing organ-bath solution into high-K⁺(30 mM) Krebs solution (in mM: 118 NaCl, 4.7 KCl, 2.55 CaCl₂, 1.18 MgSO₄, 1.18 KH₂PO₄, 24.88 NaHCO₃ and 11.1 glucose).

After stable tension was obtained, compounds were added into organ baths cumulatively (10⁻⁸M−10⁻⁴ M). The effects of compounds were expressed as a percentage of the maximum relaxation produced by 10⁻⁴ M papaverine as 100%. 50% relaxation concentration (IC₅₀) was calculated and IC₅₀ value range (μM) of compounds of the present invention was shown in the following Table 1 with a rank of A, B or C. These ranges are as mentioned below.

TABLE 1
3 μM ≧ C > 1 μM ≧ B > 0.5 μM ≧ A

| Test Compound | IC₅₀ value range |
|---|---|
| Example 4 (2) | A |
| Example 4 (3) | B |

### Experimental example 2

### Inhibitory Effect on the Rhythmic Bladder Contractions Induced by Substance P in Anesthetized Rats

For the experiments, Sprague-Dawley female rats (9 to 12 weeks old) weighing between 200 to 300 g were used. After urethane anesthetization (subcutaneously administered with a dose of 1.2 g/kg), cannulae were placed in both right and left femoral veins. One intravenous catheter was used for administration of compounds, and the other was for the substance P (0.33 μg/kg/min) infusion. We also cannulated into ureter to pass urine. Polyethylene catheters were inserted into carotid artery for continuous monitoring of arterial blood pressure and heart rate. For continuous infusion, transurethral bladder catheter was inserted into the bladder through the urethra and tied in place by a ligature around the urethral orifice. One end of the catheter was attached to a pressure transducer in order to measure intravesical pressure. The other end of the catheter was used for infusion of saline into the bladder. After stabilization of blood pressure and heart rate and after the bladder was emptied, cystometry was performed by filling the bladder slowly with about 0.6 ml of saline. After about 10 minutes, intravenous infusion of substance P (0.33 μg/kg/min) was started for stabilization of the micturition reflex. Compounds were administered after stable rhythmic bladder contraction was obtained over 15 minutes. All compounds were dissolved or suspended in saline containing 0.5% Tween 80 for intravenous administration (0.1 ml/kg). The rhythmic contraction frequency and the intravesical pressure were observed for 35 minutes after administration of the test compound.

As a result, compounds of the present invention decreased the frequency of bladder rhythmic contraction without changing the amplitude of contraction. Also, we determined a time (minute) during which the frequency of the rhythmic contraction had been completely inhibited by administering 0.25 mg/kg of compound. A 100% inhibition time (minute) of the selected compounds of the present invention is shown in the following Table 2.

TABLE 2

| Test Compound | Time (min) |
|---|---|
| Example 1 (Compound 4) | 10.08 |
| Example 8 | 8.00 |

Also, pre-administration of iberiotoxin, a selective large conductance calcium-activated K channel blocker (0.15 mg/kg, intravenous administration) reduced inhibitory effect of the compounds of the present invention on the rhythmic bladder contraction. Thus, it is suggested from the results that the compound of the present invention or a pharmaceutically acceptable salt thereof is effective for prophylaxis and treatment of diseases such as pollakiuria, urinary incontinence, and the like through the large conductance calcium-activated K channel opening activity.

### INDUSTRIAL APPLICABILITY

The compound of the present invention or a pharmaceutically acceptable salt thereof has an excellent large conductance calcium-activated K channel opening activity, so that it is useful for a prophylactic, relief and/or treatment for pollakiuria, urinary incontinence, asthma, chronic obstructive pulmonary disease (COPD), and the like.

1. An imidazole compound of the formula (1): 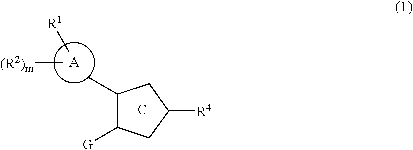
wherein Ring A is benzene or a heterocyclic ring;
G is —S(O)ₚ—R⁷, —O—R⁷, —N(R⁸)—R⁷ or 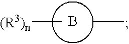
Ring B is benzene, a heterocyclic ring, a cycloalkane or a cycloalkene;
Ring C is a group selected from the following formulae: 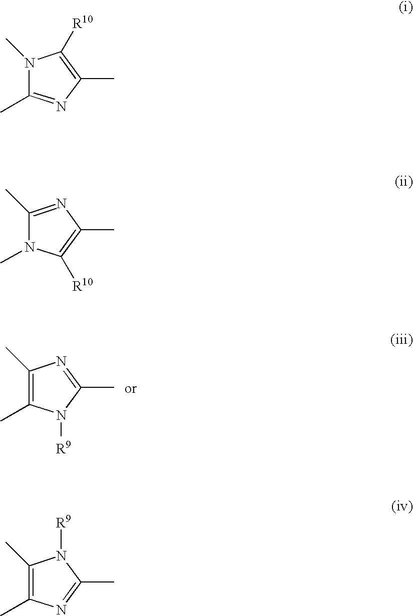
provided that when G is —S(O)ₚ—R⁷, —O—R⁷ or —N(R⁸)—R⁷, Ring C is a group of the formula (i);
R¹ is a group selected from the following formulae: 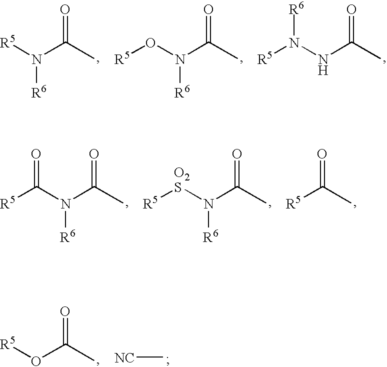
R² and R³ may be the same or different from each other, and each is cyano, nitro, hydroxyl, an optionally substituted alkoxy, a halogen, an alkanoyl, carboxy, an alkoxycarbonyl, a heterocyclic group, an optionally substituted carbamoyl, an optionally substituted amino or an optionally substituted alkyl; provided that when m is 2, two R²s may be the same or different from each other, and when n is 2, two R³s may be the same or different from each other;
m and n may be the same or different from each other, and each is 0, 1 or 2;
R⁴ is hydrogen, an alkoxy, an optionally substituted amino, an optionally substituted alkyl, an alkoxycarbonyl, an optionally substituted carbamoyl, carboxy, formyl or an optionally substituted heterocyclic group;
R⁵ and R⁶ may be the same or different from each other, and each is hydrogen, an optionally substituted alkyl, an optionally substituted cycloalkyl (wherein the cycloalkyl may be fused with an aryl), an optionally substituted aryl, an optionally substituted heterocyclic group, or an alkoxycarbonyl, or R⁵ and R⁶ may form an optionally substituted heterocyclic ring in combination with atom(s) to which they are bonded;
R⁷ is an optionally substituted alkyl, an optionally substituted aryl or an optionally substituted heterocyclic group;
p is 0, 1 or 2;
R⁸ is hydrogen or an alkyl;
R⁹ is hydrogen or an alkyl, or R⁴ and R⁹ may be combined to form an alkylene; and
R¹⁰ is hydrogen or an alkyl;
or a pharmaceutically acceptable salt thereof.

2. An imidazole compound of the formula (1a) or a pharmaceutically acceptable salt thereof according to claim 1, 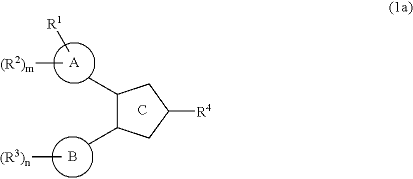
wherein Ring A, Ring B, Ring C, R¹, R², R³, R⁴, m and n have the same meanings as defined in claim 1.

3. The imidazole compound or a pharmaceutically acceptable salt thereof according to claim 1, wherein Ring A is benzene, pyridine, pyrimidine, thiazole, oxazole or thiophene.

4. The imidazole compound or a pharmaceutically acceptable salt thereof according to claim 1, wherein Ring B is benzene, pyridine, pyrimidine, thiazole, thiophene, quinoline, pyrrole, benzo[b]thiophene, thieno[2,3-b]pyridine, thieno[3,2-b]pyridine, 1,4-benzodioxane, piperidine, oxazole or cyclohexene.

5. The imidazole compound or a pharmaceutically acceptable salt thereof according to claim 1, wherein Ring B is a five-membered aromatic heterocyclic ring.

6. The imidazole compound or a pharmaceutically acceptable salt thereof according to claim 1, wherein Ring B is thiophene.

7. The imidazole compound or a pharmaceutically acceptable salt thereof according to claim 1, wherein Ring A and Ring B may be the same or different from each other and each is benzene or pyridine.

8. The imidazole compound or a pharmaceutically acceptable salt thereof according to claim 1, wherein R¹ is a group selected from the following formulae: 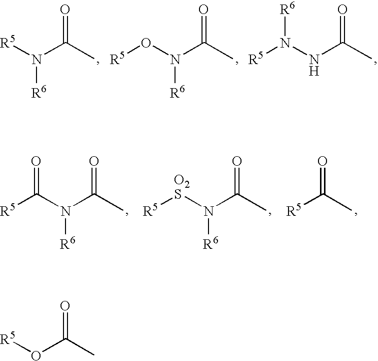
wherein R⁵ and R⁶ have the same meanings as defined in claim 1.

9. The imidazole compound or a pharmaceutically acceptable salt thereof according to claim 1, wherein R¹ is a group of the following formulae: 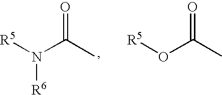
wherein R⁵ and R⁶ have the same meanings as defined in claim 1.

10. The imidazole compound or a pharmaceutically acceptable salt thereof according to claim 1, wherein R¹ is a group of the following formula: 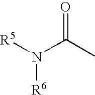
wherein R⁵ and R⁶ have the same meanings as defined in claim 1.

11. The imidazole compound or a pharmaceutically acceptable salt thereof according to claim 1, wherein R⁶ is hydrogen, an alkoxycarbonyl or an alkyl which may be substituted by hydroxy or an alkoxy, and R⁵ is hydrogen or an alkyl which may be substituted by the same or different 1 to 3 groups selected from the following formulae: 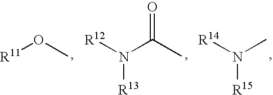
optionally substituted heterocyclic group
wherein R¹¹ is hydrogen, an alkyl or a hydroxyalkyl; R¹² and R¹³ may be the same or different from each other, and each is hydrogen, an alkyl, a hydroxyalkyl or an alkoxyalkyl; and R¹⁴ and R¹⁵ may be the same or different from each other, and each is hydrogen, an alkyl, an alkoxycarbonyl, an alkanoyl or an optionally substituted heterocyclic group.

12. The imidazole compound or a pharmaceutically acceptable salt thereof according to claim 1, wherein m and n may be the same or different from each other, and each is 0 or 1.

13. The imidazole compound or a pharmaceutically acceptable salt thereof according to claim 1, wherein R² and R³ may be the same or different from each other, and each is an alkoxy, a halogen, an optionally substituted alkyl or an optionally substituted amino.

14. The imidazole compound or a pharmaceutically acceptable salt thereof according to claim 1, wherein R⁴ is a substituted alkyl optionally substituted by 1 to 3 halogens.

15. A medicine composition comprising the imidazole compound or a pharmaceutically acceptable salt thereof according to claim 1 and a pharmaceutically acceptable carrier.

16. A method of treatment or prophylaxis comprising administering to a patient in need of treatment or prophylaxis of a disease against which a large conductance calcium-activated K channel opening activity is efficacious an effective amount of the imidazole compound or a pharmaceutically effective salt thereof according to claim 1.

17. The method according to claim 16, which is for the prophylaxis and/or treatment of pollakiuria, urinary incontinence, asthma or chronic obstructive pulmonary diseases.
